(19) United States
(12) Patent Application Publication
(10) Pub. No.: US 2026/0349479 A1
(43) Pub. Date: Nov. 5, 2026

(54) SYSTEM FOR BIOMECHANICALLY OPTIMIZING USE OF MOBILE DEVICES

(71) Applicant: UpBright LLC, Simpsonville, SC (US)

(72) Inventors: Shawn LaBelle, Simpsonville, SC (US); Thomas Lee Steiner, Simpsonville, SC (US)

(73) Assignee: UpBright LLC, Simpsonville, SC (US)

(21) Appl. No.: 19/738,382

(22) Filed: Jul. 11, 2026

Related U.S. Application Data
(63) Continuation of application No. 19/171,206, filed on Apr. 5, 2025, now Pat. No. 12,783,018.
Application No. 19/171,206 is a continuation of application No. 18/425,191, filed on Jan. 29, 2024, now Pat. No. 12,292,768.
Application No. 18/425,191 is a continuation of application No. 18/330,981, filed on Jun. 7, 2023, now Pat. No. 11,927,993.
(60) Provisional application No. 63/354,739, filed on Jun. 23, 2022.

(51) Int. Cl. G06F 1/16; G06F 1/3218

(52) U.S. Cl. CPC G06F 1/1694; G06F 1/3218; G06F 2200/1637

(57) ABSTRACT

A system for monitoring and modifying mobile device usage behavior is provided, comprising a portable computer device having an inertial measurement unit, a risk score engine computing a risk score from orientation data, an intervention module delivering outputs mapped to the risk score, a reinforcement learning agent updating intervention parameters from observed reorientation responses, and a sensor subsystem exclusively restoring outputs upon detecting orientation within a safe usage angle. Also provided is a system for training a population model, in which devices extract anonymized behavioral data via differential privacy and secure aggregation to a training module that trains cohort models using federated learning, and a distribution module initializes device parameters that transition from population-determined to individually-determined as usage data accumulates. Also provided is a system in which an active use detection module confirms active use from screen, touch, motion, and camera signals before risk score computation engages.

### RELATED APPLICATIONS

This application is a continuation in part from United States Patent Application 19/171,206 filed Apr. 05, 2025 which is a continuation from United States Patent Application 18/425,191 filed Jan. 29, 2024 which is a continuation of United States Patent Application 18/330,981 filed Jun. 07, 2023 which claims priority from United States Provisional Patent Application 63/354,739 filed Jun. 23, 2022, each of which is incorporated by reference.

### BACKGROUND OF THE INVENTION

### 1. Field of the Invention

This invention is directed to the detection and notification of aggravation of muscular pain in the head, neck, and spine due to the changes in the natural head weight bearing ability of the body resulting from improper positioning of the head and neck due to using or viewing mobile devices. Included in this functionality is the improvement to the analysis of the weight-bearing ability of the head and the negative effects brought upon improperly positioning of the head. The primary function of this invention is to modify the user’s behavior in using the mobile device to reduce the strain on the body caused by long periods of use.

### 2. Description of the Related Art

Posture, when used to describe the human body, is the position of the body in relation to gravity when standing, sitting, and laying. It is known that having a good posture ensures that the body is symmetrical in alignment and that the muscular tension is correct. Good posture involves training your body to stand, walk, bend, sit, and lay in a manner where the least amount of pressure is placed on muscles and ligaments when stationary, moving, or performing exercise activities. Benefits of a good posture include keeping bones and joints properly aligned, reducing stress on the body, reducing wear on joints, muscles, nerves, and ligaments, preventing neck, back, and muscle pain, reducing the risk of muscular fatigue, preventing the spine from becoming fixed into abnormal positions and reducing unnecessary pressure on the nerves and ligaments of the neck and spine.

When the head is in an upright position (defined by the ear being over the shoulders when viewed from the side), the neck muscles and ligaments are balanced and do not create abnormal tension of the muscles or nerves. Even a lightweight mobile device gets heavy with extended use, increased mobile device use has caused users to spend more time with their head in a forward and flexed position. This causes the relative weight of the head to shift forward. Over time this causes the head to shift more forward at rest. This phenomenon has been called “Forward Head Posture.” When the head moves forward, the head loses the support of the spinal column and now the head feels like it weighs more, dependent on how far forward the head rests. For example, a 12-pound head can feel much like a 60-pound head when the head is flexed forward to 60 degrees. Studies have shown that the position of the head forward also causes increased muscle activity of the muscles in the back of the neck that connect the skull to the upper back, especially the upper trapezius, erector spinae and neck extensor muscles. This leads to pain, stiffness and burning in those muscles from overuse. Furthermore, as the head moves forward over prolonged periods, the increased weight increases ligament lengthening in the back of the neck, while also shortening the ligaments in the front of the neck. Ligaments are designed in layers or ‘laminations.’ The nature of these laminations of ligament causes load placed on them to be translated at a slower than expected rate. This phenomenon, known as ‘hysteresis,’ causes the ligament to elongate or shorten at a slower rate than expected. The overlap of the tissues allows for a unique combination of a strong response to abrupt pulling forces, while also allowing for slow elongation over time for slow pulling forces. This is known as ligament ‘creep’. As load is placed on the ligament, it retains an initial elastic response. After a period of time, (typically 5 minutes or more, but is also dependent on the length and thickness of the ligament) the ligament begins to deform or lengthen. When someone uses a mobile device and the head is held forward for extended periods of time (greater than 5 minutes), the neck ligaments deform to shorten in the front and lengthen in the back. The neck muscles in the back become fatigued and can begin to contract, firing off mechanoreceptors, nerve sensors that relay positional sense back to the brain. This can cause contraction and spasm of the head, jaw, neck and shoulder muscles as well as the localized blood vessels, which are also under muscular control. This, in turn can fire off nociceptors, (pain sensors) which can lead to headaches, as well as pain in the neck, shoulder, jaw and arms. This can also lead to a loss of perception of normal head placement, further allowing the users head to flex forward. This can also lead to balance dysfunction. Furthermore, this forward head position causes flexion of the upper thorax and can lessen normal lung function. This cascading effect has been described as “Tech Neck.” As of January 2023, nearly 7 billion smart phones are in use worldwide. The wide acceptance of the smart phone, tablet, and other mobile devices has created patterns of use that result in “Tech Neck.” Usage research indicates that the 18-25 age cohort spends approximately four months per year engaged with displays, predominantly at approximately 30-60 degrees forward pitch from vertical.

It has been suggested that for viewing smart phones and other mobile devices, that the user should develop proper posture and neck alignment by positioning the body so that a straight vertical line can be imposed from the ear to the shoulder. While most users tilt their chin down to read a mobile device, this is not optimal. The user should raise the device to eye level so that the head isn’t perpetually dropping; an action which can result in muscle strain and injury.

Having a device that can compute a device usage risk score from orientation data to assess potentially harmful device positions would be advantageous.

It would also be advantageous if such device could deliver proportional intervention outputs, such as display luminance reduction, haptic feedback, and audio output, each scaled through a monotonically non-decreasing function of the computed device usage risk score.

It would also be advantageous for a system to restore intervention outputs automatically and exclusively upon a sensor subsystem detecting device orientation within a safe device usage angle, without conditioning restoration on any user interaction subsystem event.

It would also be advantageous for a system to include a reinforcement learning agent that actively updates intervention policy parameters based on observed individual device reorientation response outcomes, enabling adaptive behavioral intervention that reduces required intervention frequency as device usage habits form.

It would also be advantageous for such a system to receive orientation data from multiple sources including a mobile device IMU, connected wearable devices such as wrist-worn, head-worn, and ear-worn devices, and front-facing camera geometric computation, enabling multi-source sensor fusion for improved orientation measurement.

It would also be advantageous for such a system to support privacy-preserving population learning through differential privacy mechanisms and secure aggregation, enabling cohort-specific model training while protecting individual user data.

It would also be advantageous for such a system to be integrated with the operating system of the user device to facilitate ease of installation, maintenance, and interactions.

### SUMMARY

The above objectives are accomplished by providing a system for monitoring mobile device usage patterns and modifying mobile device usage behavior. The system includes a portable computer device in communication with a device IMU and adapted to receive sensor data from the device IMU. The system includes a set of computer readable instructions included on the portable computer device and configured to modify display luminance, deliver haptic feedback, deliver audio output, and any combination thereof. The system includes a device usage risk score (DURS) computation engine included in the portable computer device and configured to receive orientation data and compute a composite device usage risk score. The system includes a proportional intervention module included in the portable computer device and configured to deliver one or more intervention outputs each mapped to the composite device usage risk score through a monotonically non-decreasing function. The system includes a reinforcement learning agent included in the portable computer device configured to provide updates to intervention policy parameters based on observed individual device reorientation response, wherein the reinforcement learning agent is configured to produce measurable reduction in required intervention frequency as device usage habits form toward a safe device usage angle.

According to other aspects of the present disclosure, the system may be grounded in a Device-Gaze Inference Model that establishes device orientation as a validated proxy for inferred biomechanical risk. The system may measure orientation of the portable computer device as a validated proxy for inferred biomechanical risk without direct measurement of user anatomy. The set of computer readable instructions may be system-level software deployed on the portable computer device possessing persistent access to device IMU sensor data without application-layer background execution restrictions, authority to modify the display luminance of any foreground application without user permission at the time of modification, authority to deliver haptic and audio output during any foreground application state, and access to connected device orientation sensor data through OS-native wearable communication frameworks. The set of computer readable instructions may record the activation state of a device usage monitoring feature in association with a persistent user identifier that survives device replacement. The DURS computation engine may be configured to receive orientation data from at least one of the device IMU according to OS-native sensor frameworks, a front-facing camera adapted to provide geometric orientation information without image storage, a connected device adapted to provide inertial or orientation sensor data including wrist-worn devices, head-worn devices, and ear-worn devices accessible through OS-native wearable communication frameworks, a remote DURS computation source, and any combination thereof. When head-worn device orientation sensor data is available, the inference chain from measured orientation to inferred cervical load may shorten from three inference steps to two inference steps by eliminating the gaze-coupling inference step. The DURS computation engine may compute a continuously updated composite device usage risk score as a weighted function comprising at least current orientation angle deviation from a personalized safe device usage angle (SDUA), current episode duration, a non-linear angle-duration interaction term, a compliance history modifier, and at least one contextual modifier derived from individually observed device usage behavioral patterns, wherein the contextual modifier adapts from accumulated individual behavioral evidence and is not a fixed predetermined parameter determined at system design time. The system may include an active use detection module configured to confirm active portable computer device use by satisfaction of at least two of active screen illumination state, touchscreen interaction within a recency window, accelerometer motion pattern consistent with active handheld use as distinguished from incidental device movement, surface placement, or pocket carriage, and front-facing camera detection of a human face using OS-native face detection wherein facial image data is processed for presence detection only and not retained. The proportional intervention module may deliver all three intervention modalities simultaneously, including display luminance reduction, haptic feedback, and audio output, each independently mapped to the continuously computed DURS through a monotonically non-decreasing function. Intervention output restoration may be triggered exclusively and automatically by a sensor subsystem detecting device orientation within the SDUA, the restoration trigger not being conditioned on any input from a user interaction subsystem. The reinforcement learning agent may be configured to update intervention policy parameters based on observed individual device reorientation response outcomes including response latency, duration of maintained SDUA-compliant orientation, and rate of change in intervention frequency. The reinforcement learning agent may transition to a maintenance mode upon detecting reduction in unsafe device angular exposure frequency exceeding a statistically defined threshold over a minimum fourteen-day observation window, and may return to an active correction mode upon detecting regression.

According to other aspects of the present disclosure, the system may include one or more of the following features. The proportional intervention module may deliver all three intervention modalities simultaneously, including display luminance reduction, haptic feedback, and audio output, each independently mapped to the continuously computed DURS through a monotonically non-decreasing function. The DURS computation engine may be implemented on a connected device separate from the mobile device, wherein the connected device computes the DURS from inertial or orientation sensor data reflecting the mobile device's angular position relative to gravity and transmits the computed DURS to the mobile device's proportional intervention module for intervention output execution. The active use detection module may confirm active mobile device use by satisfaction of at least one of the four conditions. Individual intervention parameters may be determined by a population-parameter module receiving cohort-specific parameters from a remote server representing aggregated anonymous device usage statistics, without performing on-device reinforcement learning. The personalized SDUA module may be constrained such that the SDUA clinical floor for users aged 18 and above does not exceed the device pitch angle value established by peer-reviewed biomechanical research as the boundary above which inferred effective cervical load substantially increases, and wherein this floor cannot be overridden by user preference or system settings. The DURS computation engine may be configured to receive orientation data simultaneously from two or more of the mobile device IMU, a wrist-worn connected device IMU accessible through an OS-native wearable communication framework, a head-worn or ear-worn connected device IMU accessible through an OS-native audio or wearable communication framework wherein when this source is available the inference chain shortens from three inference steps to two inference steps by eliminating the gaze-coupling inference step, and a front-facing camera computing geometric orientation features without image storage, wherein the computation engine applies dynamic confidence weighting to each available source based on signal quality, connectivity state, and motion context, and wherein the system gracefully degrades to any subset of available sources without interrupting DURS computation. The active use detection module may suppress DURS computation during walking gait detected by periodic accelerometer amplitude patterns, vehicular transit detected by high-frequency vibration combined with GPS velocity, horizontal sleep position detected by sustained near-zero motion with horizontal gravity vector, and active voice call detected by OS telephony state API. The DURS computation engine may maintain a daily cumulative unsafe device angular exposure value computed as the integral of device pitch angle deviation multiplied by a non-linear weighting function, and a lifetime cumulative value representing total angle-weighted exposure since installation, and may maintain separate device usage models for vertical and horizontal device orientations with reference frame selected automatically from the gravity vector without user input. The personalized SDUA module may initialize at the clinical floor, apply population-derived initial parameter estimates as a personalization offset, and progressively update from accumulated individual device usage evidence subject to a statistical significance criterion. The system may further include an application whitelist module exempting specified applications from DURS computation and an AI-assisted whitelist suggestion module identifying applications with historically low DURS values and presenting them as whitelist candidates requiring affirmative user selection. Intervention output restoration may be triggered exclusively and automatically by the sensor subsystem detecting device and user orientation within the SDUA, and no user interface action, touchscreen input, hardware button, voice command, software instruction, confirmation dialog, or any other user interaction subsystem event of any kind conditions, gates, or serves as a restoration mechanism at any point in the restoration sequence. The reinforcement learning agent may observe device reorientation response quality comprising elapsed time from DURS threshold exceedance to SDUA reorientation, duration of maintained SDUA-compliant orientation, and statistical consistency with the user's historical compliance pattern in comparable usage contexts. The reinforcement learning agent may transition to Maintenance Mode upon detecting reduction in unsafe device angular exposure frequency exceeding a statistically defined threshold over a minimum fourteen-day observation window, and may return to Active Correction Mode upon detecting regression. The population training system includes a training module and a distribution module. The training module receives the aggregated data from the secure aggregation server and applies federated learning to train cohort-specific intervention models, without accessing any individual device's raw data. The distribution module sends the trained cohort parameters to individual devices to initialize their SDUA parameters. Each device then updates its own SDUA over time as it accumulates individual usage data, so personalization shifts from population-determined to individually-determined without any explicit mode switch or user action. Trained cohort model parameters may be returned to devices as initial SDUA and DURS parameters, transitioning automatically from population-determined to individually-determined personalization as individual device usage data accumulates. The DURS computation engine may apply age-stratified SDUA parameters comprising a pediatric clinical floor SDUA as a non-negotiable minimum sensitivity for users below a statutory minor age threshold, a parental control interface permitting authorized adults to adjust sensitivity above the floor, and data handling for users under 13 in compliance with applicable child privacy statutes.

According to another aspect of the present disclosure, a computer-implemented method for optimizing mobile device usage behavioral intervention is provided. The method includes monitoring mobile device and connected device orientation through system-accessible sensor data from at least one of the mobile device IMU, a wrist-worn device IMU, a head-worn device IMU, an ear-worn device IMU, a front-facing camera geometric computation, or a remote computation source. The method includes delivering one or more intervention outputs each scaled through a monotonically non-decreasing function of a continuously computed Device Usage Risk Score derived from orientation data and individually learned usage context parameters that are not fixed predetermined parameters. The method includes restoring all intervention outputs automatically and exclusively upon sensor subsystem detection of device and user orientation within the Safe Device Usage Angle, not conditioned on any user interaction subsystem event. The method includes actively updating, by a reinforcement learning agent, an intervention policy based on observed individual device reorientation response outcomes driven by individual user response data. The method includes automatically transitioning from active correction mode to maintenance mode upon sustained multi-week reduction in unsafe device angular exposure, and returning to active correction mode upon regression.

According to another aspect of the present disclosure, a method for training a population model for personalized mobile device usage intervention is provided. The method includes extracting on each of a plurality of mobile devices an anonymized behavioral feature vector comprising device orientation usage statistics, DURS distribution statistics, and intervention response outcome statistics, excluding raw sensor data, image data, audio data, precise location coordinates, and personally identifiable information. The method includes applying differential privacy mechanisms with a privacy parameter bounding re-identification risk. The method includes transmitting via secure aggregation wherein the server obtains only a cryptographic aggregate and cannot compute individual device contributions. The method includes training cohort-specific device usage intervention models using a federated learning algorithm configured for heterogeneous non-IID data distributions. The method includes distributing trained cohort model parameters to devices for initializing individual SDUA and DURS parameters.

### BRIEF DESCRIPTION OF FIGURES

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 is a flowchart of aspects of the system;

FIG. 2 is a block diagram of aspects of the system;

FIG. 3 is a flowchart of aspects of the system;

FIGS. 4A and 4B are illustrations of aspects of the system;

FIGS. 5A and 5B are schematic views of aspects of the system;

FIGS. 6A and 6B are schematic views of aspects of the system;

FIG. 7 is an illustration of aspects of the system;

FIG. 8 is a flowchart of aspects of the system;

FIG. 9 is a flowchart of aspects of the system;

FIG. 10 is a flowchart of aspects of the system;

FIG. 11 is a flowchart of aspects of the system;

FIG. 12 is a flowchart of aspects of the system;

FIG. 13 is a block diagram of aspects of the system;

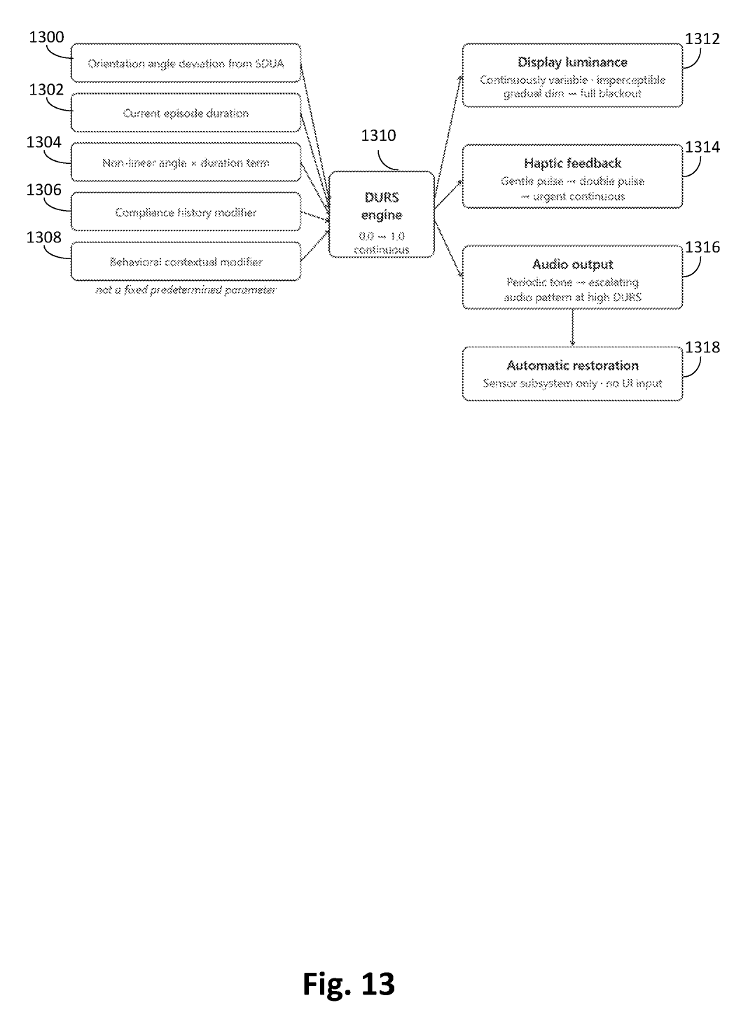

FIG. 14 is a diagram of aspects of the system;

FIG. 15 is a flowchart of aspects of the system;

FIG. 16 is a diagram of aspects of the system;

FIG. 17 is a diagram of aspects of the system; and

FIG. 18 is an illustration of aspects of the system.

While each of the drawing figures depicts a particular embodiment for purposes of depicting a clear example, other embodiments may omit, add to, reorder, and/or modify any of the elements shown in the drawing figures. For purposes of depicting clear examples, one or more figures may be described with reference to one or more other figures, but using the particular arrangement depicted in the one or more other figures is not required in other embodiments. The drawings and schematic representations are intended to support the understanding of the invention. These may not be to scale and are not intended to limit the invention to any particular layout, connectivity, or architectural implementation. Correspondence between drawing elements and described components is provided for illustrative purposes and should not be interpreted to limit the claim scope.

### DETAILED DESCRIPTION

With reference to the drawings, the invention for biomechanically optimizing mobile device use will now be described in more detail. This system, including its implementation in an operating system, improves the current hardware and software of mobile devices. Currently, mobile devices has led to “tech neck” which can be reduced or eliminated by implementation of the present system. In fact, the current operation of mobile devices created the solution that this present system solves. The system may be deployed as a system-level software component on a mobile device possessing specific capabilities that distinguish it from application-layer implementations. In some aspects, the system-level software component may possess persistent access to device IMU sensor data without application-layer background execution restrictions, enabling continuous orientation monitoring regardless of which applications are running in the foreground. The component may possess authority to modify the display luminance of any foreground application without requiring user permission at the time of modification, allowing intervention outputs to be delivered seamlessly during any device usage context. The component may possess authority to deliver haptic and audio output during any foreground application state, providing multi-modal intervention capabilities that operate independently of application-layer constraints. The component may possess access to connected device orientation sensor data through OS-native wearable communication frameworks, enabling multi-source sensor fusion with wrist-worn devices, head-worn devices, and ear-worn devices. In some embodiments, the component may record the activation state of the device usage monitoring feature in association with a persistent user identifier that survives device replacement, allowing user preferences and learned behavioral parameters to persist across device upgrades. In some aspects, this persistent user identifier may also serve as an integration point with a liability-documentation architecture.

The system disclosed herein is grounded in a Device-Gaze Inference Model that establishes the inferential basis for using device orientation as a proxy for biomechanical risk without requiring direct anatomical measurement. The Device-Gaze Inference Model takes advantage of three empirically supported relationships. First, during active device use, user gaze direction couples to the display surface orientation, as users visually attend to on-screen content. Second, sustained downward gaze beyond approximately 15 degrees is achieved primarily through forward head flexion rather than eye rotation alone, according to oculomotor research establishing physiological limits on comfortable downward eye movement. Third, forward head flexion produces quantifiable cervical load increases following a non-linear relationship documented in biomechanical research, where neutral position corresponds to approximately 12 pounds of effective cervical load, 15 degrees of flexion corresponds to approximately 27 pounds, 30 degrees corresponds to approximately 40 pounds, 45 degrees corresponds to approximately 49 pounds, and 60 degrees corresponds to approximately 60 pounds. By chaining these three relationships, the system infers biomechanical risk from device pitch angle measurement alone, enabling the DURS computation engine to assess cervical strain without cameras pointed at the user, wearable sensors attached to the body, or any direct measurement of user anatomy. This Device-Gaze Inference Model constitutes the foundational justification for the orientation-based risk assessment approach employed throughout the system.

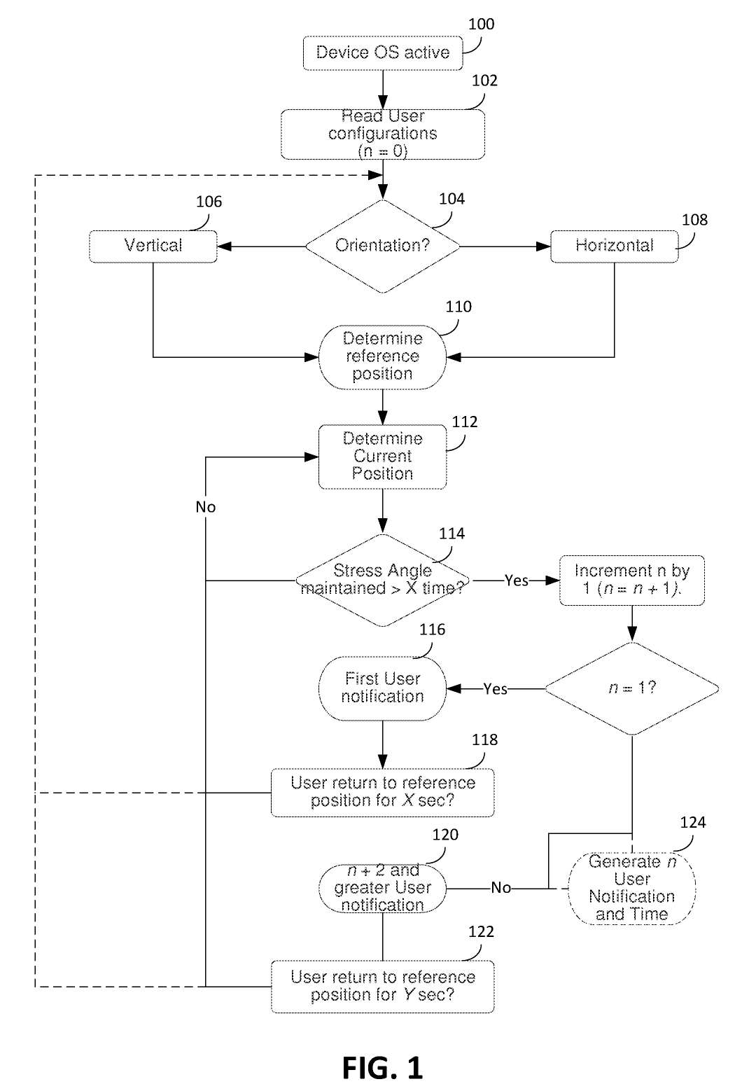

Referring to FIG. 1, the operating system and the processes, operations, and functionality that is achieved by the related hardware and software is shown. An operating system is the machine-readable language that supports computer functions such as executing applications and controlling hardware. The operating system can be included in a mobile device so that the operating system is activated at 100 when the mobile device is powered on. This embodiment does not require the user to activate an application. The operating system can have a background component that initiates upon powering of the mobile device or can be activated by the user. The background component can run with or without the user interface or otherwise with a user notification. Therefore, the operating system can capture information, receive input, and store information without the need for executing an application. The operating system can access a set of user configurations in respect to position, time, cycle iterations and other user preferences at 102.

The operating system can receive input from components of the mobile device such as the camera, magnetic field sensors, gravity sensors, location sensors (e.g., GPS receiver), accelerometers, timing device, and any combination. These inputs can be passed to an orientation determination system to determine the special orientation of the mobile device relative to the ground or the user. In a preferred embodiment, the inputs can be used to determine the positional orientation 104 of the device, where horizontal 108 is parallel to the plane of the horizon and vertical 106 is at a right angle to the horizontal 108 plane. In the vertical position 106 the user may be standing, sitting, leaning, or any combination with the spine generally perpendicular to the horizon. In the horizontal position 108, or the face down position, the user may be laying, reclining, or any similar position where the spine is approximately parallel to the horizon. Once the device determines the vertical or horizontal orientation a reference position is established at 110. The reference position is the benchmark position for proper angling of the device which all future measurements can be compared against until a new reference position is set. Additional readings may be taken from time to time to determine a current position of the mobile device as of the readings, and such readings may be triggered by a change in the orientation of the device or other movement. In some aspects, the reference position or Safe Device Usage Angle (SDUA) may represent the device pitch angle at which inferred biomechanical load is within the range documented as associated with reduced cumulative cervical strain per published research.

One or more inputs can be used to determine if a proper device position is maintained at 114 by comparison of inputs to the reference position determined at 110 and the current position determined at 112. Detection of an improper device position relative to the reference position indicates the user of the mobile device is in a potentially disadvantageous or harmful position or motion. In one embodiment, the present position of the device can be compared to the reference position to determine if the device is not within an allowable tolerance of the reference position. When outside of tolerance, the device is held at a stress angle where the user’s head is likely positioned forward of the spine causing stress to the muscles and ligaments of the neck, head, and spine. In one embodiment the operating system can determine that the mobile device is tilted at a predetermined angle. Such predetermined angle can be 10 degrees or more relative to vertical or otherwise indicated that the neck of the user is pointed downward at an undesirable angle, e.g., a stress angle. In a further embodiment the angle can be 5 degrees or more. In another embodiment, the predetermined angle can be 5 degrees or more from the horizontal, indicating the neck of the user is tilted with the chin pointed towards the chest, e.g., a stress angel. In one embodiment, position 112 supports a determination that the angular difference between the current position and the reference position exceeds the stress angle indicating the position of the mobile device is harmful to the user. The operating system can determine when an improper device position is maintained, the date, time, location, duration, and magnitude and compare the duration against a predetermined threshold at 114.

Based upon the determination at 114, the operating system can activate a user notification (e.g., reaction or response) at 116. If so, the user notification can be used to give a notification to the user. The user notification can take several forms such as dimming the screen of the mobile device, obscuring the screen, flashing the screen or other light of the mobile device, emit an audible signal, actuate a haptic assembly (e.g., vibrate or other feedback), as well as any combination. The user notification can be activated by the operating system that communicates with the mobile device hardware to change the appearance of the screen. The appearance of the screen can be modified through the operating system and can control the hardware directly such as drivers that are in communication with an operating system layer. This notification informs the user that the motion or position of the device should be changed or is undesirable.

When a notification occurs, the user can respond by modifying the motion or position of the device. The operating system can detect and record such modification including the date, time, duration, and time from notification to modification, characteristics of the modification and any combination. For example, the user can tilt the mobile device upward (typically straightening the neck) in response to the notification. Upon detecting modification of sufficient measurement for a recovery period of time of sufficient duration, the operating system can remove the user notification at 118. In one embodiment, the device is then return to the determination of orientation 104. A recovery period of time is the time interval necessary for the muscles to recover, ligaments to relax, and the body to generally recover from a stress angle. If the user continues to hold the device in a position that exceeds the predetermined angle for a user configured predetermined length of time (e.g., threshold time), a second or subsequent user notifications can be sent at 120. This cycle can be repeated with subsequent user notifications of continued failure to modify the device to an orientation within the predetermined angle repeated again at 124 when the predetermined length of time is again exceeded Thereafter the cycle from 112 to 124 can be repeated again ad infinitum. Removal of notification occurs upon modification of the device position at 122 and the sequence can return to orientation 104. Such functionality requires a time determining system comprising a means to record initial tilt exceeding the predetermined angle, a means to record duration of time during which the tilt of the device exceeds the predetermined angle, a means to reset the duration when the tilt is returned to an angle within the predetermined angle, and a means to prompt user notification and reset the duration.

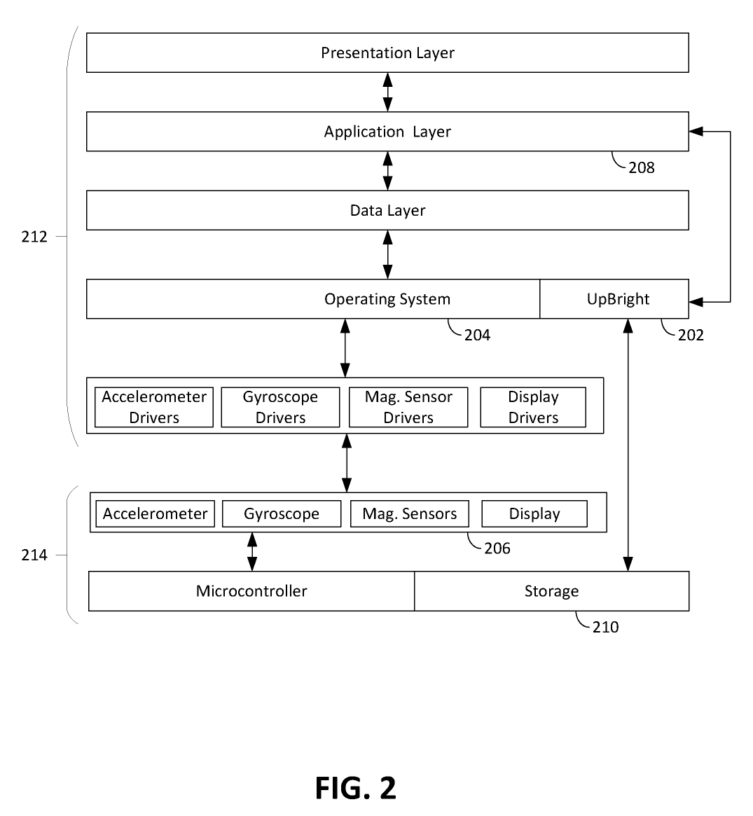

Referring to FIG. 2, computer readable instructions 202 can be included at the operating system layer 204 so that the computer readable instructions are loaded and operating when the mobile device is powered on. The operating system layer 204 can access computer readable storage 210 and user saved preferences. The operating system can use sensors 206 to detect orientation and determine an initial position or state that is an acceptable position of the mobile device. The operating system can determine when the mobile device’s position changes. This determination can be made without regard to the current applications that may be running on application layer 208. The operating system can determine that a notification is required and provide this information to any application that may be running. The notification can occur regardless of any application running as it can be determined at the operating system layer if the change in position of the mobile device warrants a notification. Because the position of the mobile device can be determined at the operating layer, the functionality of the present system does not rely upon an application, and each running application can be informed of the modifications to the mobile device, such as the screen dimming, and respond accordingly. In one embodiment, the computer readable instructions can be included in operating system 212 and executed by hardware 214. It should be noted that one or more layers, as illustrated, can be added or omitted. In some aspects, the computer readable instructions may be configured to modify display luminance by interfacing with display controller application programming interfaces (APIs) accessible through the operating system layer. The instructions may access display driver interfaces to adjust brightness levels, control backlight intensity, or apply dimming effects to the display output. In some aspects, the computer readable instructions may be configured to deliver haptic feedback by interfacing with haptic engine APIs provided by the operating system. The instructions may communicate with haptic actuator drivers to generate tactile responses including vibration patterns, pulse sequences, or continuous vibration outputs of varying intensity. In some aspects, the computer readable instructions may be configured to deliver audio output by interfacing with audio output APIs accessible through the operating system layer. The instructions may access audio driver interfaces to generate tones, alerts, or other audible signals through the device speakers or audio output hardware. The computer readable instructions may invoke these hardware control interfaces through system-level calls that bypass application-layer restrictions, enabling the instructions to modify display luminance, deliver haptic feedback, and deliver audio output during any foreground application state without requiring user permission at the time of modification.

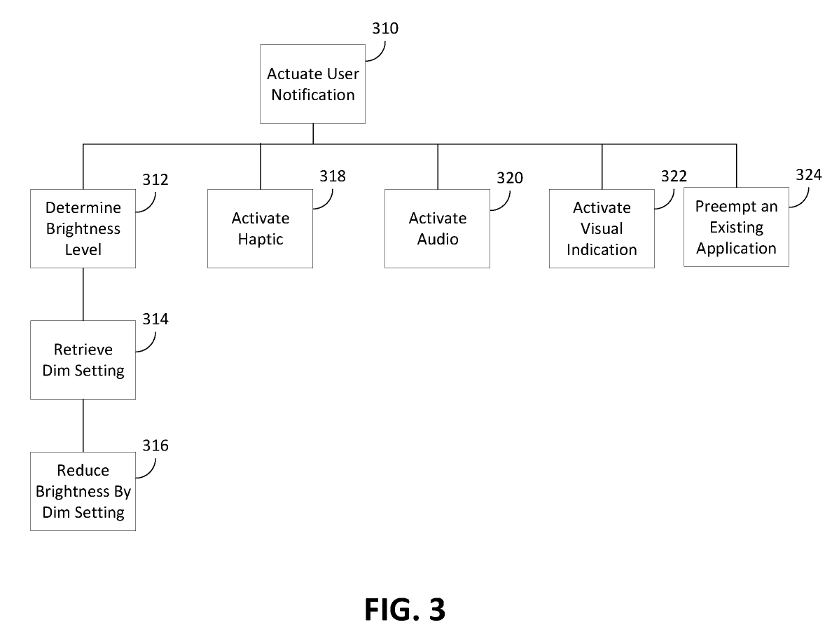

Referring to FIG. 3. The user notification 310 can include a dimming effect accomplished by determining the current screen brightness level of the mobile device at 312, retrieving the notification parameters at 314 and applying the notification at 316. For example, the operating system can determine that the current screen brightness level is 80%, retrieve that the notification is to dim the screen down to 75% of the current brightness level and reduce the brightness to 60% (on a 0 to 100 scale – 60% of 80%). The notification can be haptic at 318, audible at 320, visual at 322, and preempt or override the appearance of an executing application at 324 and any combination. In one embodiment, notification may include interruption or termination of one or more user applications. In one embodiment, the system can compare notification events and based upon the increase or decrease in frequency of notifications, amplify, or reduce the effect. For example, if the system is set to actuate notification 5 seconds after the detecting of improper position, multiple notifications can result in the actuation time being lowered to, for example, 3 seconds. Therefore, the user’s behavior has a higher chance of being modified. The modifications to the notification can include volume, brightness, time between trigger and effect, time of the effect, and any other modifications to notifications to alert the user to practice proper posture. In one embodiment, the duration of the notification can be based on a predetermined activation period of time which may be programmed into the system, user configurable, remotely configurable, or any combination.

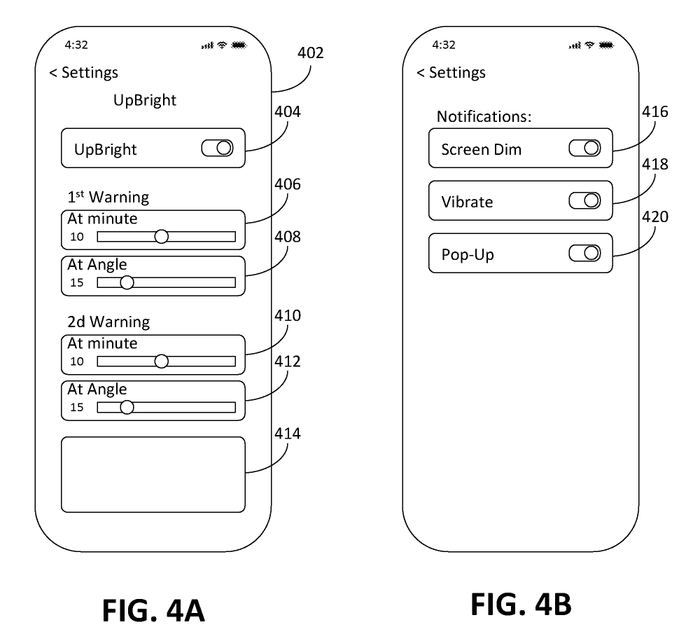

Referring to FIG. 4A, the user configurable controls of the mobile device 402 can include: a setting to discontinue use of the notification system at 404; set the initial user notification time, e.g., the predetermined length of time, at 406 and angle, e.g., the predetermined angle, at 408; set subsequent notification times at 410 and angles at 412 and personalize the notifications at 414.

Referring to FIG. 4B, further user configurable controls of the mobile device 402 can include selecting notifications such as toggles for dimming the screen at 416, vibration at 418, and pop-up notifications at 420. Further user-configured controls are possible including more detailed controls of notification, warning settings, etc.

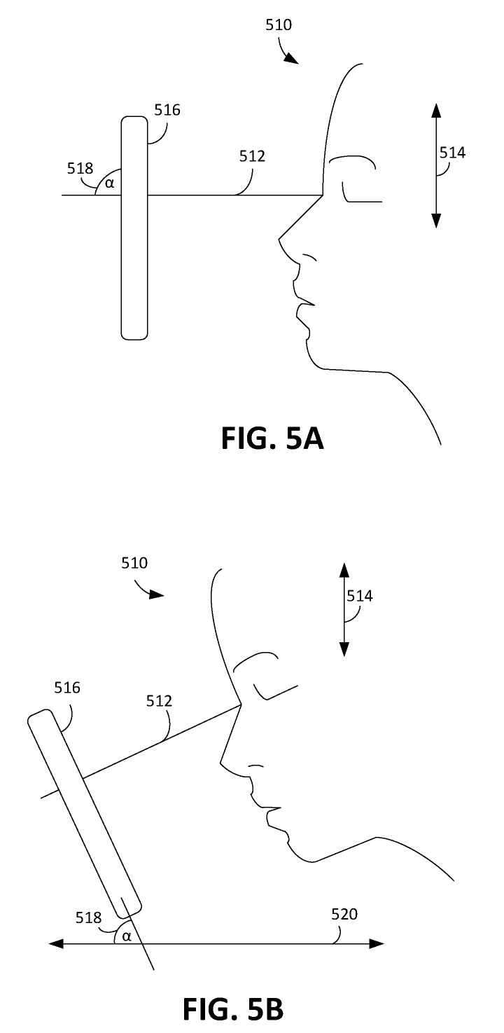

Referring to FIG. 5A, user 510 aligns the head vertically so that the line of sight 512 is generally parallel to plane 516. This position places the head at about a 90-degree angle vertically as compared with plane 514. The mobile device 516 is advantageously held at about a 90-degree angle to horizontal as shown by angle 518. FIG. 5A is an example of proper posture generally healthy for the user.

Referring to FIG. 5B, the mobile device 516 is held at less desirable angle 518 from the horizontal plane 520. While the mobile device 516 is generally at a 90-degree angle to the user’s gaze 512, the position of the user’s head is undesirable. The user 510 is shown with a disadvantageous position wherein the head is both forward and flexed when compared with the vertical plane 514. This is not optimal spinal alignment. This angle would cause the triggering mechanism of the operating system to send a user notification. The user notification may be varied in duration or intensity according to the size of stress angle 518.

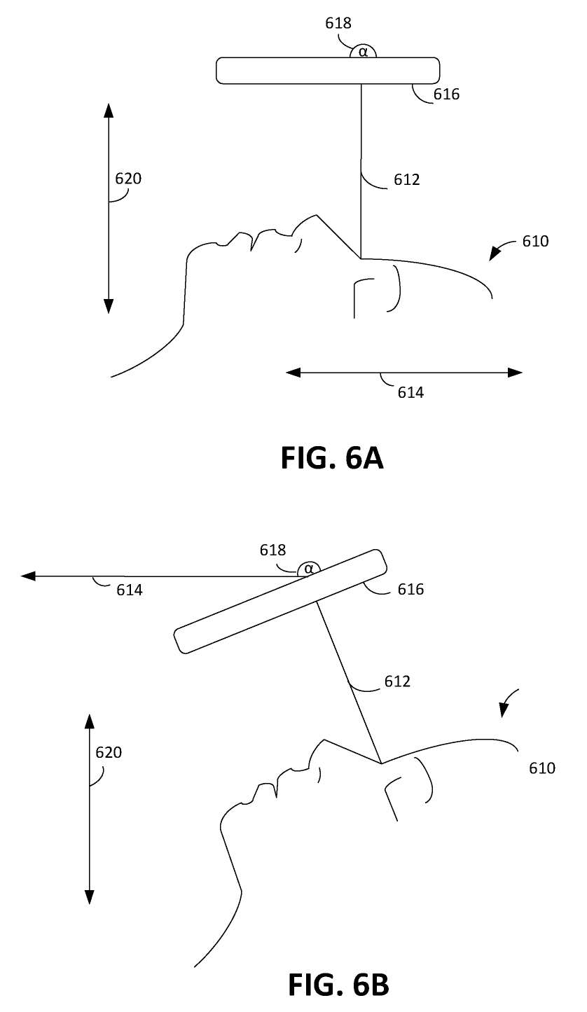

Referring to FIG. 6A, user 610 aligns the head horizontally as if the user is reclining as in lying on the floor. The line of sight 612 (e.g., user’s gaze) is generally parallel to plane 620. This position places the head at about a 180-degree angle horizontally as compared with plane 614. The mobile device 616 is advantageously held at about a 180-degree angle (e.g., approximately parallel) to horizontal as shown by angle 618.

Referring to FIG. 6B, the mobile device 616 is held at an angle 618 from the horizontal plane 614 that is less desirable. While the mobile device 616 is generally at a 90-degree angle to the user’s gaze 612, the position of the user’s head is undesirable. The user 610 is shown with a disadvantageous position wherein the head is both forward and flexed when compared to the horizontal plane 614. This is not optimal spine alignment when the user is laying down. This angle would cause the triggering mechanism of the operating system to send a user notification. The user notification may be varied in duration or intensity according to the size of stress angle 618.

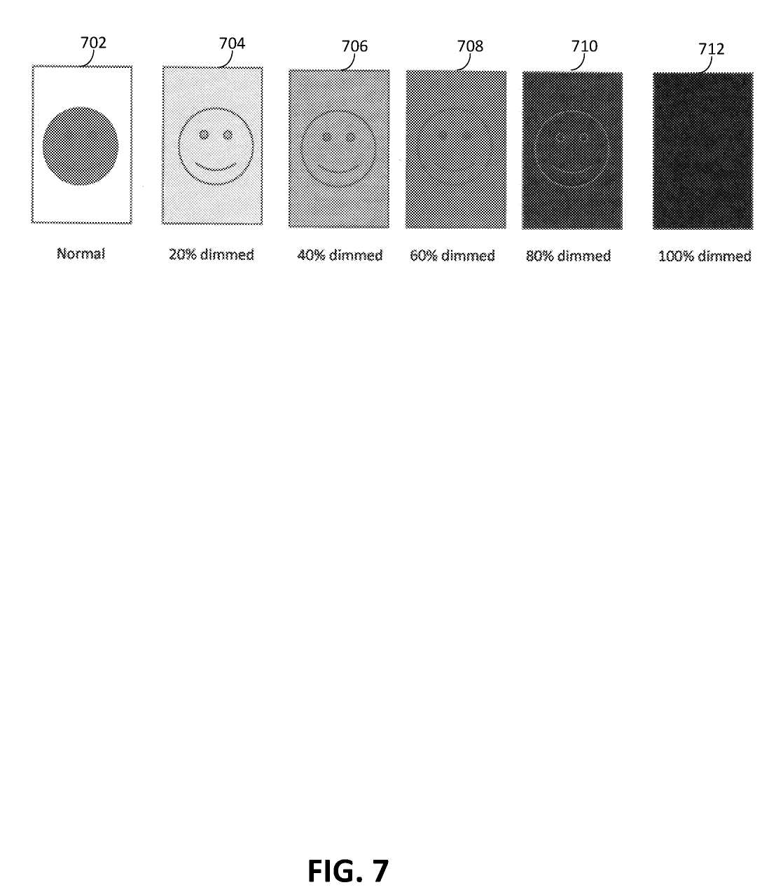

Referring to FIG. 7, the screen can be dimmed progressively according to the number of iterations of unacceptable inputs such as angles. For example, the user tilts the mobile device to an unacceptable angle multiple times in a given time period. In this case, each time the mobile device is placed in an unacceptable angle, the screen can be dimmed at a higher percentage than the previous time or for a longer time than the previous time or both. The screen dimming intensity may also be determined according to the size of the stress angle Screen 702 shows the user defined brightness when the device is held properly, Screen 704 shows the screen being dimmed by 20%. Screen 706 shows a 40% dimming effect. Screen 708 shows a 60% dimming effect. Screen 710 shows an 80% dimming effect. Screen 712 shows a 100% dimming effect. The dimming effect can be any increment between shutting off the display to no effect.

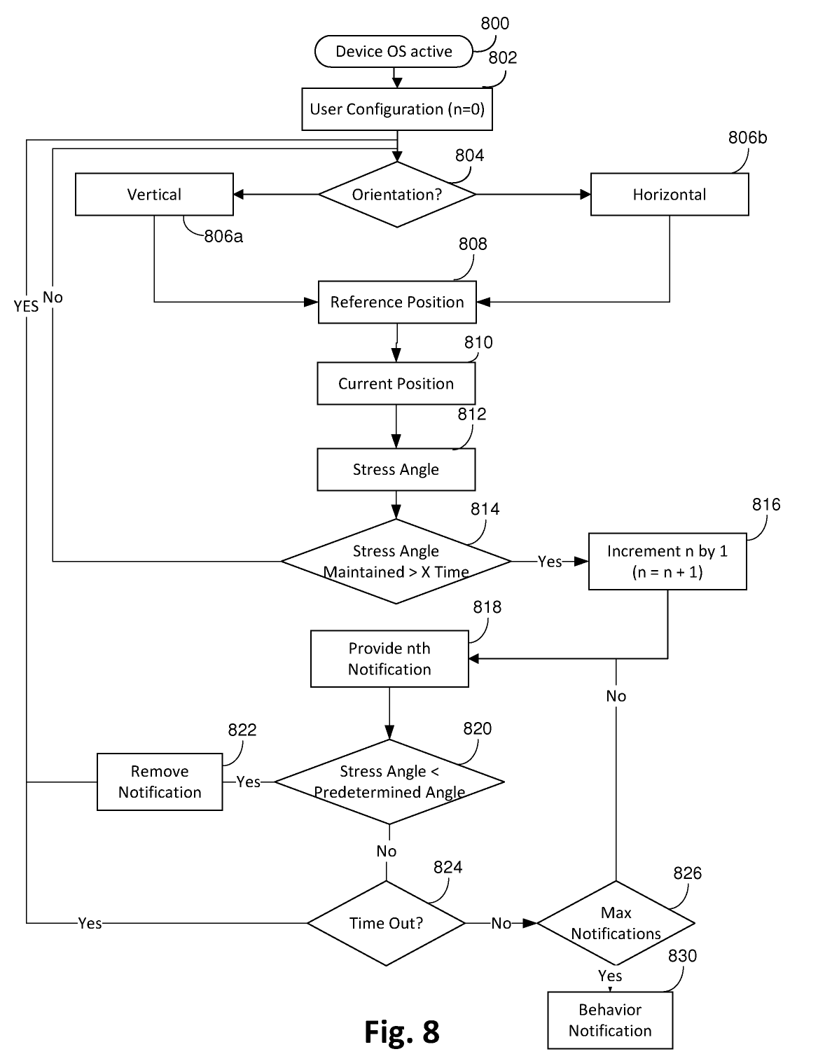

Referring to FIG. 8, one embodiment of the processes and procedures implemented with hardware and the operating system and modifications to an operating system. The operation of the system can begin with activation 800. User configuration information can be retrieved at 802 and can include setting an initial iteration counter to zero (e.g., n=0). The initial orientation of the hardware device (e.g., smart phone) can be determined at 804. The initial orientation can be classified into vertical or horizontal in one embodiment as shown in 806a and 806b respectively. According to the orientation, a reference position can be determined at 808. The reference position can be the preferred position where the portable device is positioned by the user and can be the position that minimizes or eliminates a stress angle. The position of the portable device can be determined at 810 which can be used to determine stress angle at 812. The stress angle can be the difference between the reference position and the current position. In one embodiment, when the stress angle exceeds a predetermined value, a notification can be generated by the operating system of the portable device.

In one embodiment, when the stress angle exceeds the predetermined value for length of time “X” at 814, the operating system can generate a notification. In this embodiment, notifications are generated upon prolonged periods of time when the stress angle exceeds a predetermined value rather than when the stress angle exceeds the predetermined value in passing or for small amounts of time.

In one embodiment, the operating system can increment a count of notifications at 814 and can activate a user notification at 818 to provide the user with a notification. In one embodiment the notification is achieved by dimming the screen, but such notification may also include obscuring the screen, flashing the screen or other light of the mobile device, emit an audible signal, actuate a haptic assembly (e.g., vibrate or other feedback), as well as any combination. The user notification can be activated by the operating system that communicates with the mobile device hardware to change the appearance of the screen. The appearance of the screen can be modified through the operating system and can control the hardware directly such as drivers that are in communication with an operating system layer. This notification informs the user that the motion or position of the device should be changed or is undesirable.

When a notification occurs, the operating system can determine if the user modifies the motion or position of the device so that the stress angle is less than the predetermined angle at 820. If so, the notification is removed. In one embodiment, the stress angle needs to be less than the predetermined angle for a period of time before the notification is removed.

The operating system can detect and record such modification including the date, time, duration, and time from notification to modification, characteristics of the modification and any combination. For example, the user can tilt the mobile device upward (typically straightening the neck) in response to the notification. Upon detecting modification of the position of the device for a sufficient period of time and specific duration, in one embodiment the operating system can remove the user notification at 822. In one embodiment, the operating system can determine that the stress angle exceeds the predetermined angle for an excessive period (e.g., times out) at 824 and reset the process.

In one embodiment, the operating system can rely upon a recovery period which can represent the time interval necessary for the muscles to recover, ligaments to relax, and the body to generally recover from a stress angle. If the user continues to hold the device in a position that exceeds the predetermined angle for a user configured predetermined length of time (e.g., threshold time), the operating system can take further action such as increasing the magnitude or intensive of the notification, locking the mobile device, or other notifications designed to modify the user’s behavior.

If the device is still positioned at a stress angle, the operating system can determine if a maximum number of cycles have been reached at 826. The preset maximum number of cycles may be any number including infinity where there is no maximum. If the maximum number of cycles has not been exceeded, a new cycle is started incrementing the notification count and continuing in cycle back to the cycle number determination. The cycle can be repeated with subsequent user notifications given until either the removal of notification occurs upon modification of the device position and the sequence is reset or the maximum number of cycles is reached at 826. When the maximum number of cycles is reached at 826, the system can take a behavior modification action at 808 before restoring the device path 812. The behavior modification action at 830 may require restoration to an acceptable angle relative to the reference position for a predetermined period of time to remove the effects of prior notifications. Alternatively, behavior modification action may simply be disabling the device for a period of time, limiting functionality of the device, disabling one or more applications on the device, or any similar action. The behavior modification action may be ended by passage of time, returning the device to an acceptable position, cycling the power of the device, or other such action. Such functionality requires a time determining system comprising a means to record initial tilt exceeding the predetermined angle, a means to record duration of time during which the tilt of the device exceeds the predetermined angle, a means to reset the duration when the tilt is returned to an angle within the predetermined angle, and a means to prompt user notification and reset the duration.

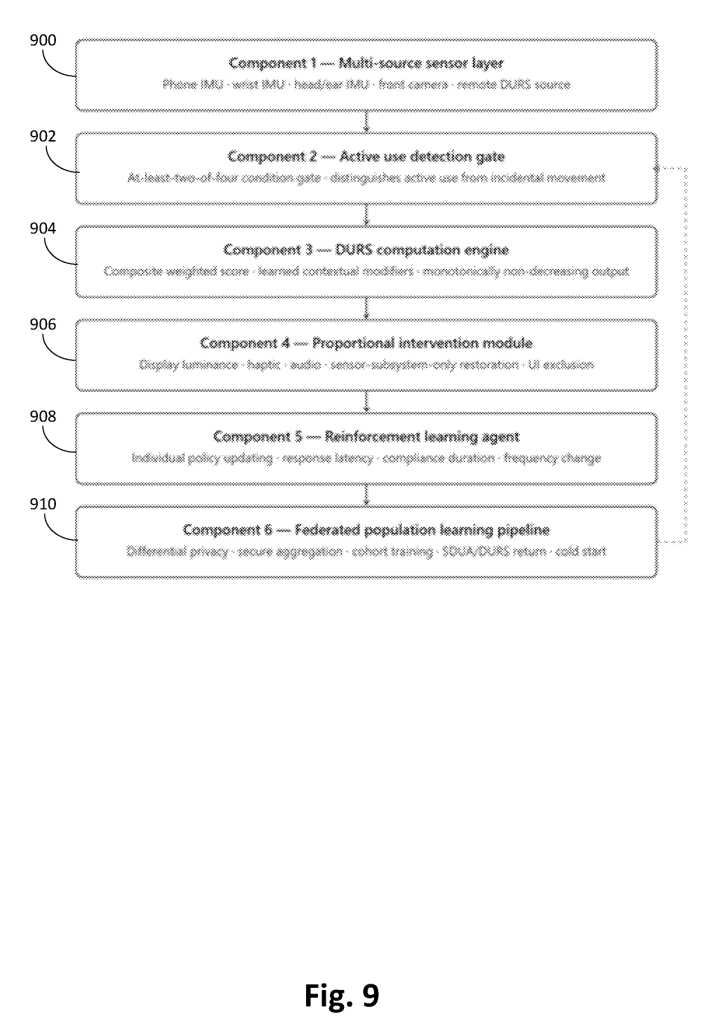

Referring to FIG. 9, a system architecture 900 for AI-driven mobile device usage pattern monitoring and adaptive behavioral intervention is shown. The architecture comprises six integrated components arranged in a sequential processing pipeline with feedback connections. A multi-source sensor layer 900 receives orientation data from multiple sources including inertial measurement units (IMUs) including a phone IMU, a wrist IMU, a head-worn or ear-worn IMU, a front camera, and a remote DURS source. IMU encompasses components such as accelerometers and gravity sensors [1] that are used to measure the orientation and movement of the mobile device. This layer serves as the data acquisition component that gathers orientation information from the mobile device and any connected wearable devices. The data from the multi-source sensor layer 900 flows to an active use detection gate 902. The active use detection gate 902 implements an at-least-two-of-four condition gate that distinguishes active use from incidental movement. This component confirms that the user is actively engaged with the mobile device rather than simply carrying or placing the device. Upon confirmation of active use, the process proceeds to a DURS computation engine 904. The device user risk score (DURS) computation engine 904 computes a composite weighted score using learned contextual modifiers and produces a monotonically non-decreasing output. This component calculates the DURS based on orientation data and individually learned behavioral parameters. The computed DURS value is then passed to a proportional intervention module 906. The proportional intervention module 906 can deliver intervention outputs including display luminance adjustment, haptic feedback, audio output, and sensor-subsystem-only restoration with user interface (UI) exclusion. This component maps the DURS value to appropriate intervention intensities. A reinforcement learning agent 908 receives feedback from the proportional intervention module 906. The reinforcement learning agent 908 performs individual policy updating based on response latency, compliance duration, and frequency change. This component actively updates intervention policy parameters based on observed individual device reorientation response outcomes. The architecture also includes a federated population learning pipeline 910, which connects to the reinforcement learning agent 908 through a feedback path. The federated population learning pipeline 910 implements differential privacy, secure aggregation, cohort training, safe device usage angle (SDUA) and DURS return, and cold start initialization. The cold start initialization includes a challenge in providing effective intervention parameters for new users who have no individual behavioral history from which to derive personalized settings. The federated population learning pipeline 910 addresses this challenge by providing cohort-derived initial SDUA and DURS parameters based on anonymous aggregate data from users with similar characteristics, enabling clinically effective intervention from day one before individual device usage data accumulates. This component enables privacy-preserving population-level learning to inform individual personalization parameters.

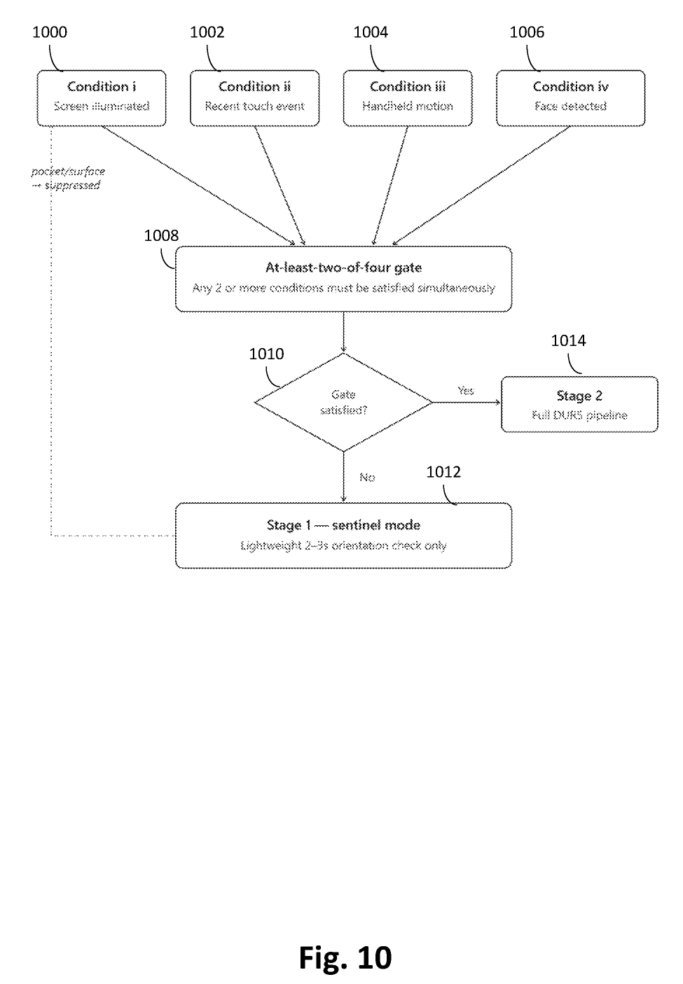

Referring to FIG. 10, an active use detection process implementing a multi-condition gate architecture is shown. The process can involve four input conditions that feed into a gate mechanism to determine whether full device usage monitoring should be activated. A step 1000 represents Condition i, which corresponds to screen illumination state detection. A step 1002 represents Condition ii, which corresponds to detection of a recent touch event on the device. A step 1004 represents Condition iii, which corresponds to detection of handheld motion patterns. A step 1006 represents Condition iv, which corresponds to face detection using the device camera. The step 1000 may include a notation indicating that pocket or surface placement conditions result in suppression of that condition. In some aspects, the recency window for touchscreen interaction detection may be a configurable time duration, such as approximately 2 to 5 seconds, within which a touchscreen interaction must have occurred for Condition ii to be satisfied. The recency window duration may be selected to balance responsiveness with tolerance for brief pauses in user interaction during active reading or viewing. In some embodiments, the recency window may be adaptive, shortening during periods of frequent interaction and lengthening during passive content consumption such as video viewing. The four conditions from step 1000, step 1002, step 1004, and step 1006 feed into a step 1008, which represents an at-least-two-of-four gate. The step 1008 requires that any two or more of the four conditions be satisfied simultaneously for the gate to be considered satisfied. This multi-condition gate distinguishes active engaged device use from incidental device movement, surface placement, or pocket carriage, and constitutes a specific technical improvement over prior systems that monitor device orientation upon any detected movement without distinguishing active engaged use from incidental movement. Following the step 1008, a step 1010 evaluates whether the gate is satisfied. If the gate is satisfied, the process proceeds to a step 1014, which represents Stage 2 involving the full DURS pipeline for comprehensive device usage risk score computation and monitoring. If the gate is not satisfied, the process proceeds to a step 1012, which represents Stage 1 sentinel mode. The step 1012 involves lightweight orientation checks occurring at approximately two to three second intervals on available sensor sources. From the step 1012, the process returns to the gate evaluation at step 1008 for continued monitoring. The flowchart depicts a hybrid two-stage architecture where the lightweight sentinel mode conserves system resources while the full DURS pipeline engages only when active device use is confirmed through the multi-condition gate mechanism.

The system may implement a hybrid two-stage architecture to balance resource efficiency with monitoring accuracy. In Stage 1, referred to as sentinel mode, the system runs lightweight orientation checks at approximately two to three second intervals on available sensor sources. This sentinel mode conserves computational resources and battery power while maintaining awareness of device orientation. When Stage 1 detects preliminary threshold exceedance and the active use detection gate confirms engaged use, the system transitions to Stage 2, referred to as active monitoring mode. In Stage 2, the full sensor fusion and DURS computation pipeline engages, providing comprehensive device usage risk assessment and proportional intervention delivery. The system returns to Stage 1 when orientation is sustained within the SDUA for a sufficient duration or when active use ends as determined by the active use detection gate. This two-stage approach allows the system to provide continuous monitoring coverage while minimizing resource consumption during periods of compliant device usage or inactive device states.

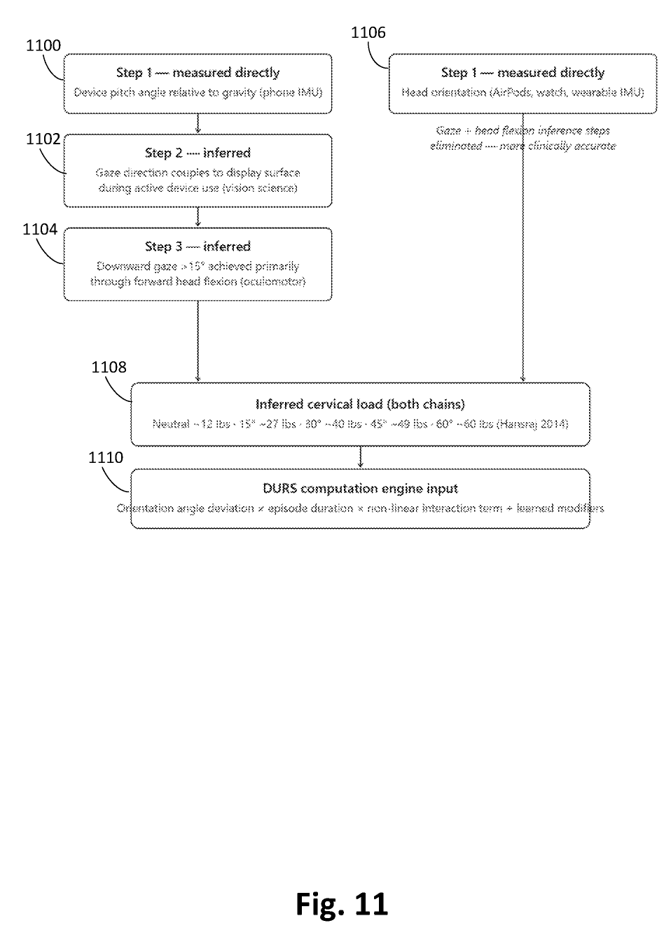

Referring to FIG. 11, the Device-Gaze Inference Model introduced above is illustrated as an inference chain comparison between phone-only orientation measurement and head-worn device orientation measurement for cervical load inference. The flowchart shows two parallel pathways that converge at a common output stage. On the left side, a step 1100 represents the first step measured directly, involving device pitch angle relative to gravity obtained from the phone IMU. The process then proceeds to a step 1102, which is an inferred step where gaze direction couples to the display surface during active device use based on vision science principles. From there, the process moves to a step 1104, another inferred step where downward gaze greater than approximately 15 degrees is achieved primarily through forward head flexion according to oculomotor research. On the right side, a step 1106 represents an alternative first step measured directly, involving head orientation that could be obtained from AirPods, watch, wearable and the like IMU. This pathway eliminates the gaze and head flexion inference steps, resulting in more clinically accurate measurement. Both pathways converge at a step 1108, which represents the inferred cervical load applicable to both chains. This step references biomechanical research indicating that neutral position corresponds to approximately 12 pounds, 15 degrees corresponds to approximately 27 pounds, 30 degrees corresponds to approximately 40 pounds, 45 degrees corresponds to approximately 49 pounds, and 60 degrees corresponds to approximately 60 pounds of effective cervical load. The process then proceeds to a step 1110, which represents the DURS computation engine input. This step calculates the Device Usage Risk Score using orientation angle deviation multiplied by episode duration multiplied by a non-linear interaction term plus learned modifiers. In one embodiment, the head-worn device pathway shortens the inference chain from three inference steps to two inference steps, where the three-step chain comprises device pitch to inferred gaze direction, inferred gaze to inferred head flexion, and inferred head flexion to inferred cervical load, and the two-step inference process that eliminates a gaze-coupling inference step comprises a first step of head-worn device orientation to inferred head orientation by virtue of physical coupling between the head-worn device and the user's head, and a second step of inferred head orientation to inferred cervical load using the biomechanical load relationship, while both pathways ultimately feed into the same DURS computation engine for proportional intervention output.

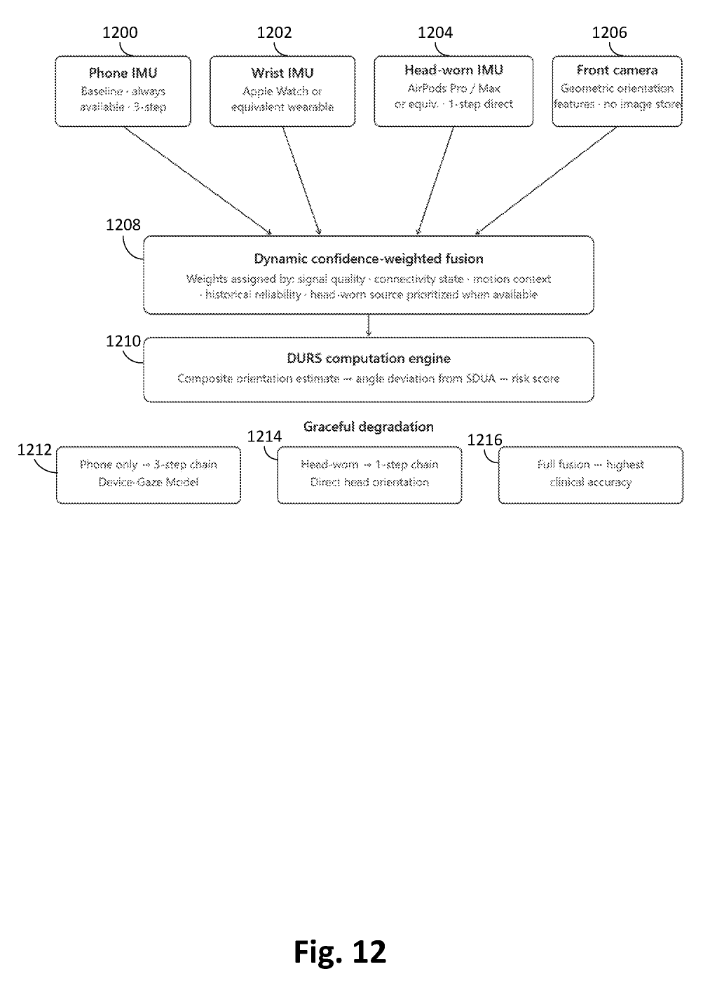

Referring to FIG. 12, a multi-source sensor fusion architecture for device usage risk score computation is shown. The architecture receives orientation data from multiple sensor sources. A phone IMU 1200 provides baseline orientation data that is always available and uses a three-step inference chain. A wrist IMU 1202 receives data from a smartwatch or equivalent wearable device. A head-worn IMU 1204 receives data from ear-worn devices or equivalent head-worn devices and provides one-step direct orientation measurement. A front camera 1206 provides geometric orientation features without image storage. The sensor data from the phone IMU 1200, wrist IMU 1202, head-worn IMU 1204, and front camera 1206 flows into a dynamic confidence-weighted fusion 1208 module. The dynamic confidence-weighted fusion 1208 assigns weights based on signal quality, connectivity state, motion context, and historical reliability, with head-worn source prioritized when available. The output from the dynamic confidence-weighted fusion 1208 feeds into a DURS computation engine 1210. The DURS computation engine 1210 generates a composite orientation estimate, calculates angle deviation from the Safe Device Usage Angle (SDUA), and produces a risk score. The architecture supports graceful degradation across three operational modes. A phone only mode 1212 operates using a three-step chain with a Device-Gaze Model when only the phone IMU is available. A head-worn mode 1214 operates using a one-step chain with direct head orientation measurement when head-worn devices are connected. A full fusion mode 1216 combines all available sensor sources to achieve the highest clinical accuracy. The system automatically selects the appropriate mode based on connected device availability and gracefully transitions between modes without interrupting DURS computation.

The input components include an orientation angle deviation from SDUA 1300, which represents the angular difference between the current device orientation and the Safe Device Usage Angle. A current episode duration 1302 tracks the length of time the device has been held at a particular orientation. A non-linear angle × duration term 1304 provides a weighted interaction between the angle deviation and the duration. A compliance history modifier 1306 incorporates historical user compliance data into the computation. A behavioral contextual modifier 1308 adapts from individually observed behavioral patterns and is not a fixed predetermined parameter, distinguishing it from static system parameters determined at design time. The DURS engine 1310 can receive these inputs and computes a continuous score ranging from 0.0 to 1.0, wherein this continuous value maps to continuously variable intervention intensity rather than discrete escalation levels.

In some aspects, the non-linear weighting function used in DURS computation and cumulative exposure calculation may take various mathematical forms. In one embodiment, the weighting function may be an exponential function of the form w(θ) = e^(k·θ) - 1, where θ represents the device pitch angle deviation from the SDUA and k is a scaling parameter that may be set to approximately 0.05 to 0.1 per degree. This exponential form assigns progressively greater weight to larger angle deviations, reflecting the non-linear relationship between head flexion angle and cervical load documented in biomechanical research. In another embodiment, the weighting function may be a polynomial function of the form w(θ) = a·θ + b·θ² + c·θ³, where the coefficients a, b, and c may be derived from curve-fitting to the empirical cervical load data. In some aspects, the weighting function may incorporate a threshold below which minimal weight is applied, such as w(θ) = 0 for θ < 5 degrees and w(θ) = (θ - 5)^1.5 for θ ≥ 5 degrees, to avoid penalizing minor deviations that do not substantially increase biomechanical risk. The specific parameters of the weighting function may be personalized based on individual user characteristics or may be derived from cohort-level training.

In some aspects, the behavioral contextual modifier 1308 may adapt from accumulated individual behavioral evidence through a continuous learning process. The system may observe and accumulate device usage behavioral patterns including time-of-day usage patterns such as morning commute sessions versus evening leisure sessions, typical session durations for different usage contexts, response latencies from intervention delivery to device reorientation, compliance rates across different environmental contexts such as home versus office versus transit, and frequency of DURS threshold exceedances during different activity types. These observed patterns may be stored in a local behavioral profile maintained on the device, with pattern statistics updated incrementally as new usage sessions occur. In some aspects, the adaptation mechanism may employ exponential moving averages that weight recent behavioral observations more heavily than older observations, allowing the modifier values to track gradual changes in user behavior over time. Alternatively or additionally, the system may employ Bayesian updating where prior beliefs about user behavior in specific contexts are updated based on accumulated observational evidence to produce posterior estimates that inform the contextual modifier values. For example, if accumulated evidence indicates that a user consistently exhibits longer response latencies during evening sessions compared to morning sessions, the contextual modifier may adapt to apply earlier or more intense interventions during evening usage contexts. The rate of adaptation may be governed by a learning rate parameter that balances responsiveness to recent behavioral changes against stability of the learned modifier values. In some embodiments, the contextual modifier may maintain separate learned parameters for distinct usage contexts identified through clustering of behavioral features, enabling context-specific adaptation that reflects the user's varying behavioral patterns across different usage scenarios.

The Safe Device Usage Angle (SDUA) represents a device orientation threshold derived from published biomechanical research. The SDUA is the target angle at which the device should be held to minimize inferred biomechanical risk to the user's cervical spine. The system monitors device orientation and delivers interventions when the device is held at angles that deviate from the personalized SDUA.

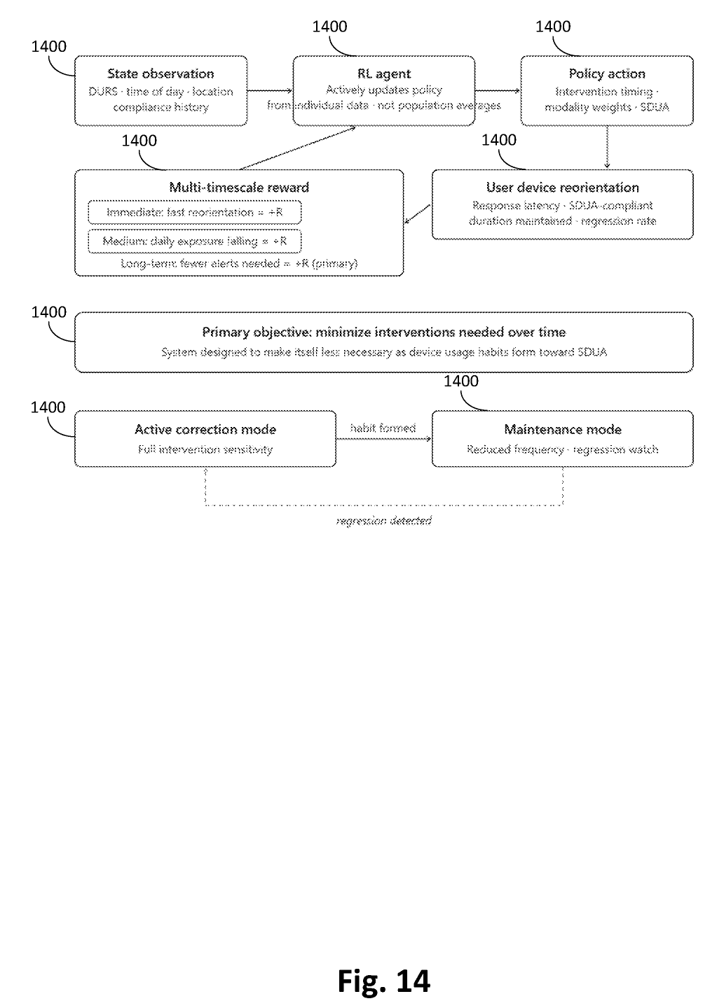

Referring to FIG. 14, a reinforcement learning system 1400 for adaptive behavioral intervention in a mobile device usage monitoring context is shown. The reinforcement learning system 1400 comprises multiple interconnected components that work together to optimize intervention policies based on individual user response data. The reinforcement learning system 1400 includes a state observation component that receives inputs including DURS, time of day, location, and compliance history. This state observation component feeds into a reinforced learning (RL) agent that actively updates policy from individual data rather than population averages. The RL agent generates policy actions that determine intervention timing, modality weights, and SDUA (Safe Device Usage Angle) parameters. The reinforcement learning system 1400 further includes a user device reorientation component that monitors response latency, SDUA-compliant duration maintained, and regression rate. This component provides feedback to a multi-timescale reward structure within the reinforcement learning system 1400. The multi-timescale reward structure may assign positive rewards across three temporal scales: immediate rewards for fast reorientation, medium-term rewards when daily exposure is falling, and long-term rewards when fewer alerts are needed over extended periods. The reinforcement learning system 1400 operates according to a primary objective of minimizing interventions needed over time. The system may be designed to make itself less necessary as device usage habits form toward the SDUA. The reinforcement learning system 1400 may transition between two operational modes: an active correction mode with full intervention sensitivity and a maintenance mode with reduced frequency and regression watch. The transition from active correction mode to maintenance mode may occur when habits are formed, while a transition back to active correction mode may be triggered when regression is detected.

In some aspects, the multi-timescale reward structure assigning rewards across immediate, medium-term, and long-term temporal scales may be implemented using weighted reward components with different temporal decay characteristics. The immediate reward component may be calculated based on response latency, such as r_immediate = max(0, 1 - t_response / t_threshold), where t_response is the elapsed time from intervention delivery to SDUA-compliant reorientation and t_threshold is a target response time such as 10 seconds. The medium-term reward component may be calculated based on daily exposure trends, such as r_medium = (E_baseline - E_today) / E_baseline, where E_baseline is the rolling average daily unsafe exposure over the previous week and E_today is the current day's cumulative unsafe exposure. The long-term reward component may be calculated based on intervention frequency reduction, such as r_long = (F_baseline - F_current) / F_baseline, where F_baseline is the average intervention frequency during an initial calibration period and F_current is the intervention frequency over the most recent observation window. In some embodiments, the composite reward may be computed as a weighted sum R = w_i · r_immediate + w_m · r_medium + w_l · r_long, where the weights w_i, w_m, and w_l may be set to values such as 0.2, 0.3, and 0.5 respectively to emphasize long-term habit formation over immediate compliance. The weights may be adaptive, with the long-term weight increasing as the user progresses toward maintenance mode. In some aspects, the reward components may incorporate exponential temporal discounting to prioritize recent behavioral evidence while maintaining sensitivity to long-term trends.

Referring to FIG. 14, a reinforcement learning system 1400 for adaptive behavioral intervention in a mobile device usage monitoring context is shown. The reinforcement learning system 1400 comprises multiple interconnected components that work together to optimize intervention policies based on individual user response data. The reinforcement learning system 1400 includes a state observation component that receives inputs including DURS, time of day, location, and compliance history. This state observation component feeds into a reinforced learning (RL) agent that actively updates policy from individual data rather than population averages. The RL agent generates policy actions that determine intervention timing, modality weights, and SDUA (Safe Device Usage Angle) parameters. The reinforcement learning system 1400 further includes a user device reorientation component that monitors response latency, SDUA-compliant duration maintained, and regression rate. This component provides feedback to a multi-timescale reward structure within the reinforcement learning system 1400. The multi-timescale reward structure may assign positive rewards across three temporal scales: immediate rewards for fast reorientation, medium-term rewards when daily exposure is falling, and long-term rewards when fewer alerts are needed over extended periods. The reinforcement learning system 1400 operates according to a primary objective of minimizing interventions needed over time. The system may be designed to make itself less necessary as device usage habits form toward the SDUA. The reinforcement learning system 1400 may transition between two operational modes: an active correction mode with full intervention sensitivity and a maintenance mode with reduced frequency and regression watch. The transition from active correction mode to maintenance mode may occur when habits are formed, while a transition back to active correction mode may be triggered when regression is detected.

In some aspects, the statistically defined threshold for transitioning to maintenance mode may comprise a percentage reduction in unsafe device angular exposure frequency, such as a 50% or greater reduction compared to a baseline period. The threshold may be evaluated using statistical hypothesis testing, such as a one-tailed t-test or Mann-Whitney U test, comparing the intervention frequency during the observation window to the baseline intervention frequency with a significance level of p < 0.05. In some embodiments, the threshold may require that the reduction be sustained with at least 95% confidence that the observed improvement is not due to random variation. The minimum fourteen-day observation window may be selected to ensure sufficient sample size for statistical validity while accounting for weekly usage pattern variations. The system may also apply a minimum effect size criterion, such as Cohen's d > 0.5, to ensure that the detected reduction represents a practically meaningful behavioral change rather than merely a statistically detectable difference.

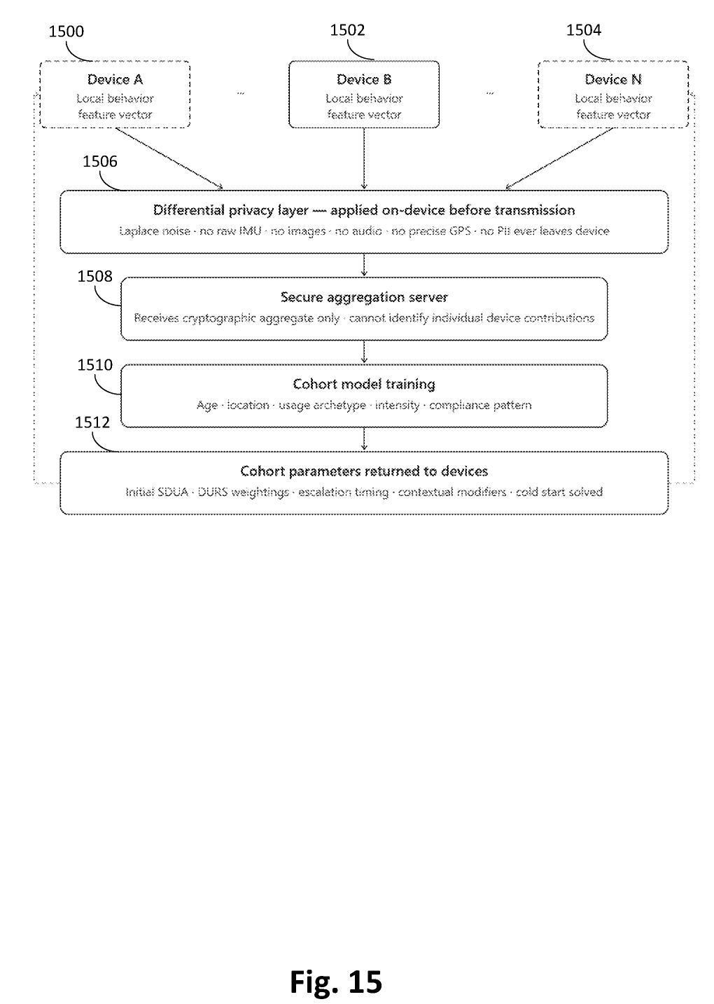

Referring to FIG. 15, a secure aggregation server 1508 is shown as a distinct component within the privacy-preserving federated learning pipeline. The secure aggregation server 1508 receives transmitted data from devices after differential privacy mechanisms have been applied by a differential privacy layer 1506. The secure aggregation server 1508 is configured such that it obtains only a cryptographic aggregate of the transmitted data and cannot compute individual device contributions. In some aspects, the secure aggregation server 1508 may implement cryptographic protocols that allow computation of aggregate statistics across multiple devices while preventing the server from accessing or reconstructing any individual device's data. The secure aggregation server 1508 passes the aggregated data to cohort model training 1510, where cohort-specific device usage intervention models are trained. The trained cohort model parameters are then distributed back to devices via cohort parameters returned to devices 1512 for initializing individual SDUA and DURS parameters.

In some aspects, the system may include a distribution module configured to distribute trained cohort model parameters to devices for initializing individual SDUA and DURS parameters. Referring to FIG. 15, the distribution module may correspond to cohort parameters returned to devices 1512, which delivers trained parameters from the cohort model training 1510 back to individual devices. The trained cohort model parameters distributed by the distribution module may include initial SDUA values, DURS weightings, escalation timing parameters, and contextual modifiers. The distribution module may communicate with devices through various mechanisms including network communication protocols, OS update channels, or background synchronization processes. In some aspects, the distribution module may address the cold start problem by providing cohort-derived initial parameters to new users who have no individual behavioral history, enabling clinically effective intervention from day one before individual device usage data accumulates. The trained cohort model parameters may transition automatically from population-determined to individually-determined personalization as individual device usage data accumulates on each device. In some embodiments, the distribution module may selectively distribute different parameter sets to different cohorts based on age, usage patterns, or other demographic or behavioral characteristics identified during cohort model training.

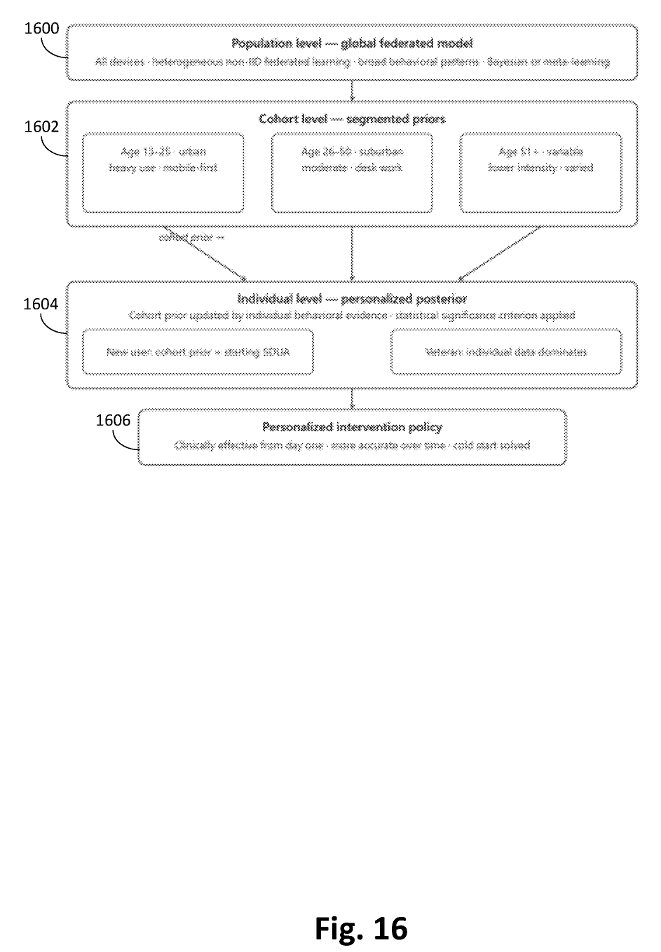

Referring to FIG. 16, a hierarchical diagram illustrating a population-to-individual personalization model for device usage intervention is shown. A population level model 1600 is positioned at the top of the hierarchy and may represent a global federated model incorporating all devices with heterogeneous non-IID federated learning and broad behavioral patterns using Bayesian or meta-learning approaches. The population level model 1600 feeds into cohort level priors 1602, which provides segmented priors organized into age-based categories. In some embodiments, these categories may include users aged 13-25 characterized as urban with heavy use and mobile-first behavior, users aged 26-50 characterized as suburban with moderate use and desk work, and users aged 51 and above characterized as variable with lower intensity and varied usage patterns. The cohort level priors 1602 flow downward into an individual level posterior 1604, which represents a personalized posterior where the cohort prior is updated by individual behavioral evidence with a statistical significance criterion applied. The individual level posterior 1604 may distinguish between new users, for whom the cohort prior serves as the starting Safe Device Usage Angle, and veteran users, for whom individual data dominates the personalization. The individual level posterior 1604 connects to a personalized intervention policy 1606 at the bottom of the hierarchy, which may deliver clinically effective intervention from day one that becomes more accurate over time, thereby addressing the cold start problem. For new users who have no individual behavioral history, the cohort-derived initial SDUA and DURS parameters provide intervention grounded in population-level behavioral evidence from the first day of use. As individual device usage data accumulates, the system transitions automatically from these population-determined parameters to individually-determined personalization, resulting in intervention that is both immediately effective and progressively more tailored to each user's specific behavioral patterns.

In some aspects, the personalized SDUA module may employ statistical methods for transitioning from population-derived parameters to individually-determined personalization. The module may implement hierarchical Bayesian inference, where the cohort-level prior distribution is updated by individual behavioral evidence to produce a personalized posterior distribution for the SDUA. Alternatively or additionally, the module may employ meta-learning approaches that learn how to adapt quickly to new users based on patterns observed across the population. Transfer learning techniques may also be applied, where knowledge gained from cohort-level training is transferred to initialize individual models that are then fine-tuned based on accumulated individual device usage evidence. The statistical significance criterion applied to SDUA updates may require that individual behavioral evidence reach a confidence threshold before overriding cohort-derived parameters, ensuring that personalization is based on sufficient data rather than noise. In some aspects, the statistical significance criterion may comprise a minimum sample size requirement, such as at least 50 usage sessions or at least 14 days of accumulated data, combined with a confidence interval threshold, such as a 95% confidence level that the individually-derived parameter differs from the cohort-derived parameter by a meaningful margin. The criterion may also include a stability requirement ensuring that the individually-derived parameter estimate has converged, such as requiring that the parameter estimate variance falls below a predetermined threshold over a rolling observation window. In one embodiment, for new users, the cohort prior serves as the starting SDUA, providing clinically effective intervention from day one. As individual data accumulates, the individual behavioral evidence progressively dominates the personalization, resulting in intervention parameters that become more accurate over time for each specific user.

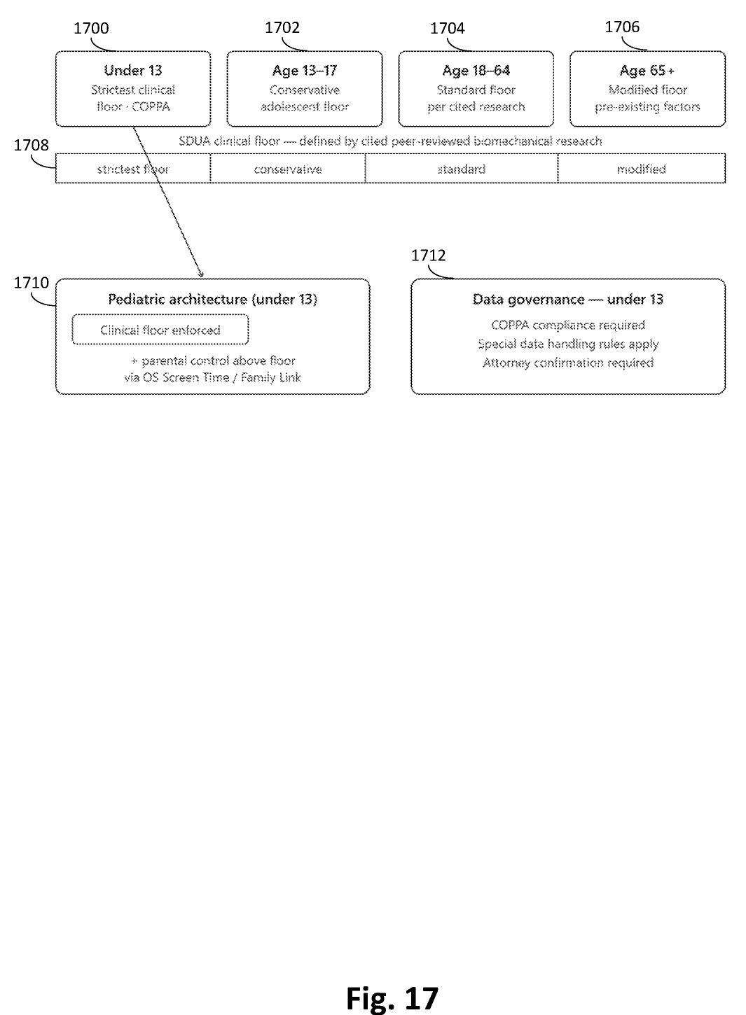

Referring to FIG. 17, an age-stratified SDUA architecture showing clinical floor constraints and data governance requirements across different age categories is shown. An under 13 age category 1700 specifies a strictest clinical floor with applicable child privacy compliance requirements. An age 13-17 category 1702 indicates a conservative adolescent floor. An age 18-64 category 1704 establishes a standard floor per cited research. An age 65+ category 1706 provides a modified floor accounting for pre-existing factors. An SDUA clinical floor 1708 spans across all age categories and may be defined by cited peer-reviewed biomechanical research, with the floor ranging from strictest for the under 13 age category 1700, to conservative for the age 13-17 category 1702, to standard for the age 18-64 category 1704, to modified for the age 65+ category 1706. In some aspects, the device pitch angle value established by peer-reviewed biomechanical research as a boundary above which inferred effective cervical load substantially increases may be approximately 15 degrees from vertical, as biomechanical research indicates that cervical load increases from approximately 12 pounds at neutral position to approximately 27 pounds at 15 degrees of forward flexion, representing a substantial increase in effective cervical load. A pediatric architecture 1710 applies to users under 13 and may enforce the clinical floor while providing parental control above the floor via OS-level parental control features. A data governance 1712 section for users under 13 may specify that applicable child privacy compliance is required and special data handling rules apply.

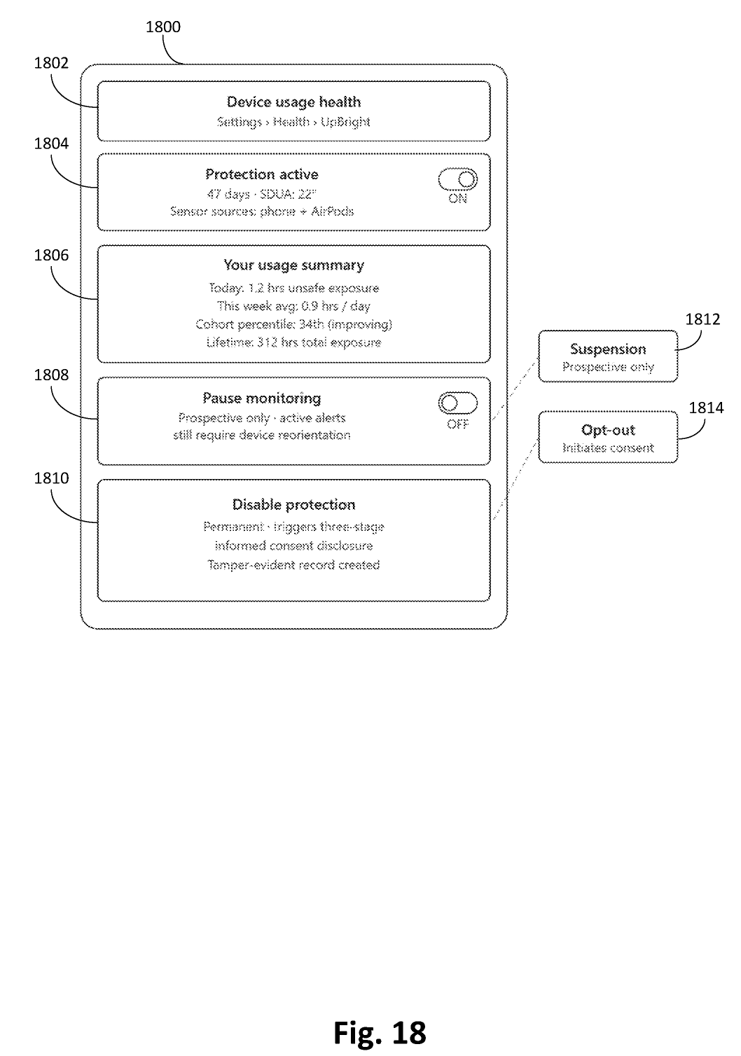

Referring to FIG. 18, a device usage health interface 1800 showing a unified settings panel 1802 for controlling device usage monitoring features is shown. The settings panel 1802 contains multiple sections arranged vertically. A protection active section 1804 displays the current monitoring status including the number of days active, the SDUA value, and the sensor sources being used such as phone and connected ear-worn devices, with a toggle switch shown in the ON position. A usage summary section 1806 presents device usage statistics including today’s unsafe exposure hours, weekly average exposure, cohort percentile ranking with improvement indication, and lifetime total exposure hours. A pause monitoring section 1808 provides a toggle switch for temporarily suspending monitoring on a prospective-only basis, with a notation that active alerts still require device reorientation. A disable protection section 1810 offers a permanent disabling option that may trigger a multi-stage informed consent disclosure and create a tamper-evident record. The interface also shows a suspension control 1812 labeled as prospective only, and an opt-out control 1814 that initiates a consent process, both positioned adjacent to the pause monitoring section 1808. In one embodiment, the monitoring suspension mode control and device usage health settings are presented within the OS Settings application. The suspension control may be clearly labeled as a time-limited prospective pause that does not affect any intervention currently in progress.

In some aspects, the monitoring suspension mode may pause DURS computation and new intervention output prospectively for a defined period but does not affect any intervention currently in progress. When a user activates the suspension control while an intervention is active, the active intervention persists until automatic sensor-detected reorientation occurs. This design preserves the exclusive sensor-based restoration mechanism even during suspension periods, ensuring that no user interface action can dismiss an active intervention. The suspension control affects only future DURS computation and new intervention initiation, not the resolution of existing interventions. In a preferred configuration, the suspension control is clearly labeled as a time-limited prospective pause that does not affect any intervention currently in progress, and the control may be presented within the OS Settings application alongside other device usage health settings. This approach maintains the integrity of the behavioral intervention system while providing users with flexibility to temporarily pause monitoring for legitimate purposes.

In some aspects, the system may include an application whitelist module that exempts specified applications from DURS computation. Certain applications may naturally encourage device positions within the SDUA due to their content type, interaction patterns, or display characteristics. For example, applications that present content in a manner conducive to upright device positioning may consistently produce low DURS values during use. The system may include an AI-assisted whitelist suggestion module that analyzes historical DURS values associated with different applications over time. The AI-assisted whitelist suggestion module may identify applications with historically low DURS values and present these applications as candidates for addition to the whitelist. In some aspects, the AI-assisted whitelist suggestion module may track DURS distributions on a per-application basis and flag applications whose usage sessions consistently fall within or near the SDUA. The module may apply statistical criteria to distinguish applications that genuinely encourage favorable device positioning from applications that merely happen to have been used during compliant periods. Before any application is added to the whitelist, the system may require affirmative user selection, ensuring that users maintain control over which applications are exempted from DURS computation. This approach allows the system to reduce unnecessary interventions during use of applications that do not pose biomechanical risk while preserving user agency over whitelist configuration.

The following representative use cases illustrate practical and concrete examples of system operation across different user scenarios. In an office worker scenario with multi-modal intervention, a 38-year-old user employs a tablet as a secondary display. The reinforcement learning agent learns from response latency data that display dimming alone is insufficient to prompt reorientation and increases the haptic weighting in the intervention policy. Over a six-week period, measured unsafe angular exposure may fall significantly as the user develops improved device usage habits. In some cases as much as 71%.

In an adolescent transit scenario, a 16-year-old commutes approximately 45 minutes daily. The pediatric clinical floor applies due to the user's age. The reinforcement learning agent determines that audio intervention is more effective than display dimming in the noisy transit environment. Over a four-week period, average device pitch angle may decrease and daily unsafe exposure hours may be reduced. In some cases this pitch angle decrease can range from approximately 52 degrees to approximately 28 degrees, and daily unsafe exposure hours may be reduced from approximately 2.1 hours to approximately 0.5 hours as device usage habits form.

In a senior reclining scenario, a 67-year-old user employs a tablet while reclining. The horizontal orientation model and senior-cohort SDUA parameters can apply. The reinforcement learning agent determines that haptic feedback produces the most responsive reorientation behavior. Over a five-week period, unsafe angular exposure may decrease as the user adapts device usage patterns. On some cases this decrease can be 58%.

In a distributed watch computation scenario, a 29-year-old user's smartwatch IMU measures wrist orientation. The connected device computes the DURS from orientation data reflecting the phone's angular position relative to gravity and transmits the computed DURS to the phone's proportional intervention module for intervention output execution, rather than transmitting raw sensor data.

In a baseline phone-only scenario, a user holds a phone at approximately 55 degrees forward pitch for 12 minutes. The system infers approximately 55 degrees below horizontal gaze direction and corresponding cervical load using the three-step inference chain. The DURS crosses the intervention threshold at approximately four minutes, and display dimming, haptic feedback, and audio output engage proportionally. When the user raises the device to within the SDUA, all intervention outputs restore automatically through sensor-detected reorientation, with no user interface action required or available.

In a head-worn device scenario, a 34-year-old user wears ear-worn devices containing IMUs used for spatial audio head tracking. The sensor fusion layer assigns high confidence to the head-worn source due to its direct physical coupling with head orientation. The inference chain shortens from three steps to two steps, and DURS accuracy improves. If the user removes the ear-worn devices mid-session, the system gracefully degrades to phone-IMU-only operation and resumes the three-step inference chain without interrupting DURS computation or intervention delivery.

As used herein, the terms "component," "module," "engine," and "agent" may include a unit implemented in hardware, software, or firmware, and may be interchangeably used with other terms such as "logic," "logic block," "part," or "circuitry." Each of these terms may indicate hardware, software, or both that provides the described functionality. For example, according to aspects of the disclosure, a module, component, engine, or agent may be implemented in a form of an application-specific integrated circuit (ASIC), a field-programmable gate array (FPGA), a digital signal processor, or software instructions executed by a processor. As used herein, the term "condition" may refer to a detectable state, criterion, or determination that is evaluated as part of a decision process, and may indicate hardware, software, or both that provides the described functionality of detecting or evaluating the state.

In the description, numerous details are set forth for purposes of explanation in order to provide a thorough understanding of the present invention. However, it will be apparent to one skilled in the art that not all of these specific details are required in order to practice the present invention. It is also noted that the invention may be described as a process, which is usually depicted as a flowchart, a flow diagram, a structure diagram, or a block diagram. Although a flowchart may describe the operations as a sequential process, many of the operations can be performed in parallel or concurrently. In addition, the order of the operations may be re-arranged.

As used herein, the term "device IMU" or "inertial measurement unit" may refer to a sensor assembly that measures orientation and movement of the portable computer device. The device IMU may include one or more of accelerometers configured to measure linear acceleration along one or more axes, gyroscopes configured to measure angular velocity or rotational motion, and magnetometers configured to measure magnetic field orientation. In some aspects, the device IMU may be implemented as a single integrated circuit package containing multiple sensor elements, such as a micro-electromechanical systems (MEMS) device that combines accelerometer and gyroscope functionality. In other aspects, the device IMU may comprise discrete sensor components mounted on the portable computer device's circuit board. The device IMU may communicate orientation data to a processor of the portable computer device through standard sensor interfaces, such as I2C, SPI, or other digital communication protocols. The orientation data from the device IMU may include raw sensor readings, processed orientation angles, or quaternion representations of device attitude. In some embodiments, the device IMU may include sensor fusion algorithms that combine data from multiple sensor types to provide more accurate orientation estimates. The device IMU may operate continuously or may be activated in response to device state changes or application requests.

As used herein, the term "sensor subsystem" may refer to the collection of hardware and software components that detect and report device orientation data to the system. The sensor subsystem may comprise the device IMU of the portable computer device, including accelerometers and gyroscopes that measure orientation relative to gravity. In some aspects, the sensor subsystem may further comprise orientation sensors of connected devices, such as wrist-worn devices, head-worn devices, and ear-worn devices, that communicate orientation data to the portable computer device through OS-native wearable communication frameworks. The sensor subsystem may detect device orientation by processing raw sensor readings from one or more of these sources and computing orientation angles, such as device pitch angle relative to gravity. The sensor subsystem operates independently of the user interaction subsystem, which comprises components that receive user input such as touchscreen interactions, hardware button presses, voice commands, and other user-initiated actions. The sensor subsystem may continuously monitor orientation and provide orientation data to the DURS computation engine without requiring any user interaction. In some aspects, the sensor subsystem may detect when device orientation returns to within the Safe Device Usage Angle and trigger restoration of intervention outputs automatically, without any input from the user interaction subsystem.

As used herein, the term "user interaction subsystem" may refer to the collection of hardware and software components that receive user input on the portable computer device. The user interaction subsystem may comprise touchscreen input components that detect and process touch gestures and taps on the device display, hardware buttons including power buttons, volume buttons, and home buttons, voice command interfaces that receive and process spoken user instructions, software-generated confirmation dialogs and user interface controls, and any other input mechanisms through which a user may provide intentional input to the device. The user interaction subsystem operates independently of the sensor subsystem, which detects device orientation and other environmental conditions without requiring user input. In the context of intervention output restoration, the distinction between the sensor subsystem and the user interaction subsystem is significant because restoration is triggered exclusively by sensor-detected orientation changes rather than by any form of user input through the user interaction subsystem.

As is known in the art, the operations described above can be performed by hardware, software, or some combination of software and hardware. Various aspects of embodiments of the invention may be implemented using circuits and logic devices (hardware), while other aspects may be implemented using instructions stored on a machine-readable medium (software), which if executed by a processor, would cause the processor to perform a method to carry out embodiments of the invention. Furthermore, some embodiments of the invention may be performed solely in hardware, whereas other embodiments may be performed solely in software. Moreover, the various functions described can be performed in a single unit or can be spread across a number of components in any number of ways. When performed by software, the methods may be executed by a processor, based on instructions stored on a computer-readable medium. If desired, the instructions can be stored on the medium in a compressed and/or encrypted format.

It is understood that the above descriptions and illustrations are intended to be illustrative and not restrictive. It is to be understood that changes and variations may be made without departing from the spirit or scope of the following claims. Other embodiments as well as many applications besides the examples provided will be apparent to those of skill in the art upon reading the above description. The scope of the invention should therefore be determined, not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventor did not consider such subject matter to be part of the disclosed inventive subject matter.

What is claimed is:

1. A system for monitoring mobile device usage patterns and modifying mobile device usage behavior comprising:
a portable computer device having an inertial measurement unit (IMU);
computer readable instructions integrated in an operating system of the portable computer device and configured to modify display luminance, deliver haptic feedback, and deliver audio output;
a device usage risk score (DURS) computation engine configured to compute a composite device usage risk score as a weighted function comprising at least a current orientation angle deviation from a personalized safe device usage angle (SDUA);
a proportional intervention module configured to deliver an intervention output;
a reinforcement learning agent configured to update intervention policy parameters based on observed individual device reorientation response outcomes; and
a sensor subsystem configured to trigger restoration of intervention outputs automatically and exclusively upon detection of device orientation within the SDUA, wherein restoration is not conditioned on any user interaction subsystem input.

2. The system of claim 1, wherein the weighted function further comprises at least one of a current episode duration and a contextual modifier derived from individually observed device usage behavioral patterns that adapts from accumulated individual behavioral evidence.

3. The system of claim 1, wherein the system measures orientation of the portable computer device as a proxy for inferred biomechanical risk without direct measurement of user anatomy, wherein the DURS computation engine computes the orientation angle deviation from the personalized SDUA based on the measured orientation, and wherein the computer readable instructions are deployed at an operating system layer of the portable computer device with persistent access to device IMU sensor data without application-layer background execution restrictions.

4. The system of claim 3, wherein the DURS computation engine infers biomechanical risk using a multi-step inference chain comprising a gaze-coupling inference step relating a device pitch angle to an inferred gaze direction, and a subsequent inference step relating the inferred gaze direction to an inferred head flexion angle.

5. The system of claim 1, further comprising a head-worn device in communication with the portable computer device, wherein the head-worn device provides head orientation measurement data enabling the DURS computation engine to infer cervical load using a two-step inference process that eliminates a gaze-coupling inference step.

6. The system of claim 5, wherein the DURS computation engine is configured to receive orientation data from at least one of: the device IMU, a front-facing camera adapted to provide geometric orientation information without image storage, a connected device including wrist-worn devices, head-worn devices, and ear-worn devices accessible through an OS-native wearable communication framework, and a remote DURS computation source.

7. The system of claim 1, wherein the proportional intervention module delivers display luminance reduction, haptic feedback, and audio output simultaneously, each independently mapped to the composite device usage risk score through a monotonically non-decreasing function to provide continuously variable intervention intensity.

8. The system of claim 1, wherein the reinforcement learning agent transitions to a maintenance mode upon detecting a reduction in unsafe device angular exposure frequency exceeding a statistically defined threshold over a minimum fourteen-day observation window, and returns to an active correction mode upon detecting regression.

9. The system of claim 1, wherein the system records an activation state of a device usage monitoring feature in association with a persistent user identifier that survives device replacement, and wherein an SDUA clinical floor for users aged 18 and above does not exceed a device pitch angle value established by peer-reviewed biomechanical research as a boundary above which inferred effective cervical load substantially increases, and wherein the clinical floor cannot be overridden by user preference or system settings.

10. The system of claim 9, wherein the SDUA is in a range of 1 to 60 degrees from vertical.

11. The system of claim 1, wherein the DURS computation engine is implemented on a connected device separate from the portable computer device, wherein the connected device computes the composite device usage risk score from inertial or orientation sensor data and transmits the computed composite device usage risk score to the proportional intervention module for intervention output execution.

12. The system of claim 1, wherein the DURS computation engine maintains a daily cumulative unsafe device angular exposure value computed as an integral of device pitch angle deviation multiplied by a non-linear weighting function, and a lifetime cumulative value representing total angle-weighted exposure since installation, and wherein the DURS computation engine maintains separate device usage models for vertical and horizontal device orientations with a reference frame selected automatically from a gravity vector.

13. The system of claim 1, further comprising an application whitelist module exempting specified applications from device usage risk score computation, and an AI-assisted whitelist suggestion module identifying applications with historically low device usage risk score values and presenting the applications as whitelist candidates requiring affirmative user selection.

14. The system of claim 1, further comprising a user configuration interface allowing a user to selectively enable or disable one or more of the intervention outputs.

15. The system of claim 1, wherein the intervention output comprises at least one of display luminance reduction, screen obscuring, screen flashing, haptic feedback including vibration patterns and pulse sequences, audio output including tones and audible alerts, visual pop-up notifications, application preemption, application interruption, application termination, device disabling, functionality limitation, and any combination thereof.

16. The system of claim 1, wherein the personalized SDUA is initialized using cohort model parameters received from the distribution module.

17. A system for training a population model for personalized mobile device usage intervention comprising:
a plurality of mobile devices, each configured to extract an anonymized behavioral feature vector comprising device orientation usage statistics;
a differential privacy module configured to apply differential privacy mechanisms to the anonymized behavioral feature vector;
a secure aggregation server configured to receive transmitted data as a cryptographic aggregate without computing individual device contributions;
a training module configured to train cohort-specific device usage intervention models using federated learning; and
a distribution module configured to distribute trained cohort model parameters to devices for initializing individual safe device usage angle parameters, wherein the parameters transition from population-determined to individually-determined personalization as individual device usage data accumulates.

18. The system of claim 17, wherein the trained cohort model parameters include age-stratified safe device usage angle parameters comprising a pediatric clinical floor as a non-negotiable minimum sensitivity for users below a statutory minor age threshold, a parental control interface permitting authorized adults to adjust sensitivity above the pediatric clinical floor, and data handling for users under 13 in compliance with applicable child privacy statutes.

19. The system of claim 17, wherein each mobile device further comprises a personalized SDUA module that initializes at a clinical floor, applies population-derived initial parameter estimates as a personalization offset, and progressively updates from accumulated individual device usage evidence subject to a statistical significance criterion.

20. A system for monitoring mobile device usage patterns comprising:
a portable computer device having a device inertial measurement unit (IMU);
an active use detection module configured to confirm active device use by at least one of an active screen illumination, a touchscreen interaction within a recency window, an accelerometer motion pattern consistent with handheld use, and a camera-based face detection;
a device usage risk score (DURS) computation engine configured to compute a composite device usage risk score upon confirmation of active use; and
a proportional intervention module configured to deliver at least one of a display luminance reduction, a haptic feedback, and an audio output, each mapped to the composite device usage risk score through a monotonically non-decreasing function.

21. The system of claim 20, wherein the active use detection module, the DURS computation engine, and the proportional intervention module are implemented at an operating system layer of the portable computer device with persistent access to device IMU sensor data without application-layer background execution restrictions.

22. The system of claim 20, wherein the active use detection module suppresses computation of the composite device usage risk score during: walking gait detected by periodic accelerometer amplitude patterns, vehicular transit detected by high-frequency vibration combined with GPS velocity, horizontal sleep position detected by sustained near-zero motion with horizontal gravity vector, and active voice call detected by an OS telephony state API.

23. The system of claim 20, further comprising a reinforcement learning agent configured to update intervention policy parameters based on observed individual device reorientation response outcomes, wherein the reinforcement learning agent is configured to produce measurable reduction in required intervention frequency as device usage habits form toward a safe device usage angle.

24. The system of claim 20, wherein restoration of the intervention outputs is triggered exclusively and automatically by a sensor subsystem detecting device orientation within a safe device usage angle, wherein restoration is not conditioned on any user interaction subsystem input.

25. The system of claim 20, wherein the DURS computation engine is configured to receive orientation data from a connected device comprising a wearable device worn on the body, attached to clothing, handheld, or otherwise carried by the user, including wrist-worn devices, head-worn devices, ear-worn devices, and clothing-attached devices.

26. The system of claim 20, wherein the DURS computation engine computes the composite device usage risk score using a non-linear angle-duration interaction term that weights a product of orientation angle deviation and episode duration, and wherein the reinforcement learning agent employs a multi-timescale reward structure assigning rewards across immediate, medium-term, and long-term temporal scales.
